(19) United States
(12) Patent Application Publication
(10) Pub. No.: US 2024/0124857 A1
(43) Pub. Date: Apr. 18, 2024

(54) COMPOSITION FOR GUT HEALTH

(71) Applicant: DUPONT NUTRITION BIOSCIENCES APS, KONGENS LYNGBY (DK)

(72) Inventors: CHARLES BUDINOFF, OREGON, WI (US); BRANDON J BURKHART, CHALFONT, PA (US); RITESH KUMAR, WILMINGTON, DE (US); ANDREW JOHN MORGAN, WILTSHIRE (GB)

(21) Appl. No.: 18/277,440

(22) PCT Filed: Feb. 18, 2022
(86) PCT No.: PCT/US2022/016924

§ 371 Date: Aug. 16, 2023

Related U.S. Application Data
(60) Provisional application No. 63/289,439, filed on Dec. 14, 2021.
(60) Provisional application No. 63/151,190, filed on Feb. 19, 2021.

(51) Int. Cl. C12N 9/16; A61K 35/745

(52) U.S. Cl. CPC C12N 9/16; A61K 35/745; C12Y 301/03002

(57) ABSTRACT

Provided herein, inter alia, are compositions and methods for treating and/or preventing conditions associated with gut inflammation caused by dysbiosis via use of exogenously administered acid phosphatases or enzymes of the GDA1_CD39 superfamily (such as apyrases).

### CROSS-REFERENCE TO RELATED APPLICATIONS

This application is a 371 of International Application No. PCT/US2022/016924, filed Feb. 18, 2022 and claims priority to U.S. Provisional Patent Application No. 63/151,190, filed Feb. 19, 2021, and U.S. Provisional Patent Application No. 63/289,439, filed Dec. 14, 2021, the disclosures of each of which are incorporated by reference herein in its entirety.

### INCORPORATION BY REFERENCE OF SEQUENCE LISTING

The present application is being filed along with a Sequence Listing in electronic format. The Sequence Listing is provided as a file entitled 20230815_NB41936USPCT_Seq_List.txt, created 15 Aug. 2023 which is 107,015 bytes in size. The information in the electronic format of the Sequence Listing is incorporated by reference in its entirety.

### FIELD OF THE INVENTION

Provided herein, inter alia, are compositions and methods for treating and/or preventing conditions associated with gut inflammation caused by dysbiosis via use of exogenously administered acid phosphatases or enzymes of the GDA1_CD39 superfamily (such as apyrases).

### BACKGROUND

Through millions of years of evolution, metazoans have developed mechanisms that maintain a mutually beneficial symbiotic relationship with commensal microbiota. Intestinal microbes play a pivotal role in maintaining human health and wellbeing (Marchesi and Shanahan, Curr Opin Infect Dis, 20:508, 2007; Blaut and Clavel, J. Nutr, 137:751S, 2007; Turnbaugh et al., Nature, 444:1027, 2006; Dethlefsen et al., Trends Ecol Evol, 21:517, 2006; Lupp and Finlay, Curr Biol., 15:R235, 2005; Backhed et al., Science, 307:1915, 2005; Mai and Morris, Jr., J. Nutr, 134:459, 2004; Falk et al., Microbiol Mol Biol Rev, 62:1157 ; 1998). Mucosa-associated bacteria, in conjunction with the mucus-layer and the epithelium, provide a direct selective barrier between the gut and systemic sites, allowing the absorption of nutrients, water, and electrolytes, while inhibiting the translocation of pathogenic microbes or toxins. The microbiota synthesizes vitamins, detoxifies toxins, and also provides additional energy for the epithelial cells by fermentation of otherwise non-digestible food components, such as fiber and starch. Commensal organisms also participate in the maturation and maintenance of the gastrointestinal tract immune system, keeping the intestinal epithelia in a state of ‘physiological inflammation’ that appears to be critical for a rapid response against potentially harmful bacteria.

However, bacterial products such as lipopolysaccharides (LPS; also referred to as endotoxins), extracellular ATP (exATP), CpG DNA, and flagellin are key components that play a vital role in mediating bacterial-induced inflammation. Dysbiosis, defined as dysregulation of the normal homeostasis of the intestinal microbiota, has been implicated in the pathogenesis of myriad inflammatory disease conditions, including, but not limited to, antibiotic-associated diarrhea (AAD), Clostridium difficile-associated disease (CDAD), acquired immunodeficiency syndrome (AIDS), hypothyroidism, food poisoning, obesity, inflammatory bowel disease (IBD), irritable bowel syndrome (IBS), and colorectal carcinoma (Dethlefsen et al., Trends Ecol Evol, 21:517, 2006; Schroeder, Am Fam Physician, 71:921, 2005; Hurley and Nguyen, Arch Intern Med, 162:2177, 2002; Bartlett, N Engl J. Med, 346:334, 2002; McFarland, Dig Dis, 16:292, 1998; Wistrom et al., J. Antimicrob Chemother, 47:43, 2001; Marteau et al., Aliment Pharmacol Ther, 20 Suppl 4:18, 2004; Seksik et al., Gut, 52:237, 2003; Kleessen, Scand Gastroenterol, 37:1034, 2002; Brenchley et al., Nat Med, 12:1365, 2006; Mai and Morris, J. Nutr, 134:459, 2004; Seksik et al., Gut, 52:237, 2003; Marteau et al., Aliment Pharmacol Ther, 20 Suppl 4:18, 2004; Kleessen et al., Scand J Gastroenterol, 37:1034, 2002; Pool-Zobel et al., Br J Nutr, 87 Suppl 2:S273, 2002).

Unfortunately, the fundamental mechanisms that govern the normal homeostatic number and composition of the intestinal microbiota remain poorly understood as well as the corresponding influence these organisms have on mediation of gut-inflammation during dysbiosis. What is needed, therefore, are compositions and methods that can be used to decrease gut inflammation brought about by dysbiosis and inflammation-causing bacterial products such as LPS, exATP, CpG DNA, and flagellin.

The subject matter disclosed herein addresses these needs and provides additional benefits as well.

### SUMMARY

Provided herein, inter alia, are compositions and methods for treating and/or preventing conditions associated with gut inflammation caused by dysbiosis via use of exogenously administered acid phosphatases or enzymes of the GDA1_CD39 superfamily (such as apyrases).

Accordingly, in some aspects, provided herein are compositions comprising a) an enzyme that hydrolyzes nucleotide triphosphates, wherein said enzyme is active at least from about pH 3.5 to pH 7; and b) an enteric coating. In some embodiments, the enzyme 1) is a member of the GDA1_CD39 superfamily; and 2)a) is not potato apyrase; and/or b) is not a mammalian nucleoside triphosphate diphosphohydrolases (NTPDase). In some embodiments, the enzyme comprises a polypeptide at least about 40% or at least about 60% identical (such as at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the amino acid sequence of any of SEQ ID NO:1, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some embodiments, the polypeptide comprises amino acid residues S204, F205, L206, G207, L208, and G209 at positions corresponding to the numbering of SEQ ID NO:9. In some embodiments, the enzyme is 1) an acid phosphatase (EC 3.1.3.2) and; 2) is not derived from Shigella. In some embodiments, the enzyme comprises a polypeptide at least about 50% or at least about 66% identical (such as at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO:2, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24. In some embodiments of any of the embodiments disclosed herein, the nucleotide triphosphate comprises ATP. In some embodiments of any of the embodiments disclosed herein, the nucleotide diphosphate comprises ADP. In some embodiments of any of the embodiments disclosed herein, the enzyme has peak specific activity on ATP from at least from about pH 4 to about pH 5. In some embodiments of any of the embodiments disclosed herein, the enzyme hydrolyzes ATP with a specific activity of at least about 500 μmol/mg/min to about 2700 μmol/mg/min from about pH 3.5 to about pH 7. In some embodiments of any of the embodiments disclosed herein, the enteric coating is for 1) oral administration; 2) delivery to the intestinal mucosa; and 3) protection from degradation of the enzyme at a pH of less than about 2.5. In some embodiments of any of the embodiments disclosed herein, the enteric coating is formulated for rectal, oral, or parenteral administration.

In further aspects, provided herein is a nucleic acid encoding any of the enzymes that hydrolyze nucleotide triphosphates disclosed herein, including a nucleic acid that is at least about 40% or at least about 50% (such as at least about 40%, 45%, 50%, 55%, 60%, 65%, 70%, 75%, 80%, 85%, 90%, 95%, or 100% (inclusive of all values in between these percentages) identical to a nucleic acid encoding any one of the polypeptides of SEQ ID NO:1, SEQ ID NO:2 or SEQ ID NOs:6-24.

In another aspect, provided herein is a vector comprising any of the nucleic acids disclosed herein. In some embodiments, the vector comprises a polynucleotide (such as a synthetic polynucleotide) that is at least about 40% (such as at least about 40%, 45%, 50%, 55%, 60%, 65%, 70%, 75%, 80%, 85%, 90%, 95%, or 100% (inclusive of all values in between these percentages) identical to any of SEQ ID NOs:25, 26, 27, 28, 29, 30, 31, 32, 33, 34, 35, 36, 37, 38, 39, 40, 41, 42, or 43.

In still further aspects, provided herein is a recombinant host cell comprising any of the enzymes that hydrolyze nucleotide triphosphates disclosed herein (including an enzyme comprising a polypeptide at least about 40% or at least about 60% identical (such as at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the amino acid sequence of any of SEQ ID NO:1, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16, or at least about 50% or at least about 66% identical (such as at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO:2, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24), any of the nucleic acids disclosed herein (such as any of SEQ ID NOs:25-43 or nucleic acids at least about 50%, 55%, 60%, 65%, 70%, 75%, 80%, 85%, 90%, 95%, or 100% identical to any of SEQ ID NOs:25-43), and/or any of the vectors disclosed herein. In some embodiments, the polypeptide comprises amino acid residues S204, F205, L206, G207, L208, and G209 at positions corresponding to the numbering of SEQ ID NO:9. In some embodiments, the cell is a plant cell, a bacterial cell, a fungal cell, or a yeast cell. In some embodiments of any of the embodiments disclosed herein, the cell is a Bacillus subtilis cell, an E. coli cell, a Yarrowia cell, an Aspergillus niger cell, or a Trichoderma reesei cell.

In other aspects, provided herein is a method for decreasing or preventing a disorder characterized by inflammation in the gut in a subject in need thereof, comprising administering to said subject any of the compositions disclosed herein (including a composition comprising a polypeptide at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of any of SEQ ID NO:1, SEQ ID NO:6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16, or at least about 50%, at least about 55%, at least about 60%, at least about 65%, at least about 66%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of SEQ ID NO:2, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24). In some embodiments, the polypeptide comprises amino acid residues S204, F205, L206, G207, L208, and G209 at positions corresponding to the numbering of SEQ ID NO:9. In some embodiments, the disorder is a gastro-intestinal (GI) tract inflammatory disease selected from the group consisting of inflammatory bowel disease (IBD), Crohn's disease, colitis, colitis ulcerosa, enterocolitis, and necrotising enterocolitis. In some embodiments of any of the embodiments disclosed herein, the gut consists of the large intestine.

In still other aspects, provided herein is a method for promoting the engraftment of one or more commensal bacteria into the gut of a subject, said method comprising administering to said subject any of the compositions comprising an enzyme that hydrolyzes nucleotide triphosphates disclosed herein (such as an enzyme at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of any of SEQ ID NO:1, SEQ ID NO:6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16 or at least about 50%, at least about 55%, at least about 60%, at least about 65%, at least about 66%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of SEQ ID NO:2, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24) and a source of one or more commensal bacteria. In some embodiments, said administration is oral administration. In some embodiments, said administration of the enzyme is oral administration and administration of the source of one or more commensal bacteria is rectal administration. In some embodiments of any of the embodiments disclosed herein, said subject has been diagnosed with a disorder characterized by inflammation in the gut. In some embodiments, the disorder is a gastro-intestinal (GI) tract inflammatory disease selected from the group consisting of inflammatory bowel disease (IBD), Crohn's disease, colitis, colitis ulcerosa, enterocolitis, and necrotising enterocolitis. In some embodiments of any of the embodiments disclosed herein, the gut consists of the large intestine. In some embodiments of any of the embodiments disclosed herein, the source of one or more commensal bacteria comprises a probiotic. In some embodiments of any of the embodiments disclosed herein, the source of one or more commensal bacteria comprises a fecal microbiota transplant (FMT). In some embodiments of any of the embodiments disclosed herein, the one or more commensal bacteria is selected from the group consisting of Ligilactobacillus ruminis, Prevotella copri, Ligilactobacillus salivarius, and Bifidobacterium longum.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 is a line graph depicting candidate enzyme specific activity (in μmol/mg/min) on ATP over a range of pHs.

FIG. 2 is a line graph depicting candidate enzyme specific activity (in μmol/mg/min) on ADP over a range of pHs.

FIG. 3 is a bar graph depicting relative activity of candidate enzymes on different nucleotide substrate relative to ATP.

FIG. 4 is a bar graph depicting residual activity of candidate enzymes after incubation at pH 2 for 15 or 60 min.

FIG. 5A and FIG. 5B depict DAI score in non-catheterized mice-Disease symptoms of colitis were assessed daily by measurement of body weight, evaluation of stool consistency, and detection of bloody stools. Disease severity was scored using a clinical disease activity index (DAI), calculated as previously described using the following parameters: stool consistency, presence or absence of fecal blood and weight loss.

FIG. 6A and FIG. 6B depict DAI score in catheterized mice-Disease symptoms of colitis were assessed daily by measurement of body weight, evaluation of stool consistency, and detection of bloody stools. Disease severity was scored using a clinical disease activity index (DAI), calculated as previously described using the following parameters: stool consistency, presence or absence of fecal blood and weight loss.

FIG. 7 depicts a schematic showing the experimental design for the treatments discussed in Example 3.

FIG. 8 depicts a bar graph showing disease activity index (DAI) among treatment groups using the following parameters: stool consistency, presence or absence of fecal blood and weight loss.

FIG. 9 depicts a bar graph showing colon length measurements among treatment

groups.

FIG. 10 depicts a bar graph showing focal hyperplasia in the colon among treatment groups assessed using histopathological analysis.

FIG. 11 depicts a series of bar graphs showing the engraftment of commensal bacteria in the guts of mice treat with acid phosphatase in combination with human microbial transplant (HMT).

FIG. 12 depicts a multiple protein sequence alignment of CRC22110 apyrase and homologs

FIG. 13 depicts a pairwise protein sequence alignment of CRC22110 and 4BRA.

FIG. 14 depicts a sequence motif that is critical for ATP hydrolysis activity in Legionella pneumophila apyrase which is adjacent to active site which and occupied by an ANP molecule (black sticks) in PDB ID: 4BRA structure.

FIG. 15 depicts a multiple protein sequence alignment performed using Clustal W including bacterial acid phosphates evaluated in this study, and other acid phosphatases: 1D2T, KEY80034.1, AAK09393.1 and AAF66599.1.

FIG. 16 depicts a phylogenetic tree for the sequences constructed using neighbor-joining method MEGA X for a set of acid phosphatase sequences to highlight the bacterial acid phosphatases group.

FIG. 17 depicts a region in acid phosphatase class A enzymes that is critical for ATP degrading activity adjacent to the active site (indicated by the phosphate molecule) from the 1D2T crystal structure.

### DETAILED DESCRIPTION

ATP signaling plays a critical role in inducing inflammation. Enzymatic removal of extracellular ATP from the microenvironment may provide a solution for treating and/or prevention of inflammation. However, this approach may require an enzyme that can function at low pH, as decreased intestinal pH has been reported to occur under inflamed gastrointestinal conditions. The invention disclosed herein provides a solution to this problem which was absent in past reported approaches for overcoming inflammation in the gastrointestinal tract.

Accordingly, this invention is based, at least in part, on the discovery that administering (e.g., orally) an acid phosphatase and/or an enzyme of the GDA1_CD39 superfamily (e.g. an apyrase), to a subject in a therapeutically effective amount, can treat or prevent inflammation associated with gut dysbiosis or pathogenic infection, e.g. colitis or irritable dowel disease (IBD), can promote the engraftment of beneficial commensal bacteria in the gut, and can thus safely and easily reduce one or more symptoms of gut tissue inflammation with little or no side effects.

### I. Definitions

The term “acid phosphatase” (EC 3.1.3.2) (also known as acid phosphomonoesterase, phosphomonoesterase, glycerophosphatase, acid monophosphatase, acid phosphohydrolase, acid phosphomonoester hydrolase, uteroferrin, acid nucleoside diphosphate phosphatase, acid phosphatase (class A) or orthophosphoric-monoester phosphohydrolase (acid optimum)) is used to mean an enzyme having a pH optimum for mediating hydrolysis of a phosphate ester bond in a substrate at a pH less than about pH 6.5, such as less than about pH 4.0. Acid phosphatases free attached phosphoryl groups from other molecules during digestion. It can be further classified as a phosphomonoesterase. Acid phosphatase is stored in lysosomes and functions when these fuse with endosomes, which are acidified while they function; therefore, it has an acid pH optimum. This enzyme is present in many animal and plant species. In some embodiments, the acid phosphatase used in the compositions and methods disclosed herein is not derived from Shigella. In some embodiments, the acid phosphatase used in the compositions and methods disclosed herein is not derived from a mammal.

As used herein, the “GDA1_CD39 superfamily refers to enzymes comprised of nucleoside triphosphate diphosphohydolases (NTPDases) with common motifs in their protein sequences. The family is named after two proteins: the yeast GDPase (GDA1) and a lymphoid cell activation antigen, CD39. These proteins, in some embodiments, are cell-surface enzymes that hydrolyze a range of NTPs, including extracellular ATP. Non-limiting examples include ecto-ATPases, apyrases, CD39s, and ecto-ATP/Dases (Knowles, 2011, Purinergic Signaling volume 7, pages 21-45, incorporated by reference herein).

The term “apyrase,” as used herein, refers to one or more of a calcium-activated enzyme (i.e. proteins belonging to class EC. 3.6.1.5) which possesses ATP-diphosphohydrolase activity and catalyzes the hydrolysis of the gamma phosphate from ATP, and catalyzes the hydrolysis of the beta phosphate from ADP. Apyrases are found in all eukaryotes and some prokaryotic organisms, indicating a preserved role for these enzymes across species. They possess a distinct phosphohydrolase activity, nucleotide substrate specificity, divalent cation requirement, and sensitivity to inhibitors. (See, Plesner, Int. Rev. Cytol., 158:141 (1995), and Handa & Guidotti, Biochem. Biophys. Res. Commun., 218(3):916 (1996)). In mammals, apyrase is believed to function primarily as an extracellular hydrolase specific for ATP and ADP, which function is important in the inactivation of synaptic ATP molecules following nerve stimulation. (See, Todorov et al., Nature, 387(6628):76 (1997)). Apyrase in mammals is also believed to be important in the inhibition of ADP-induced platelet aggregation. (See, Marcus et al., J. Clin. Invest., 99(6):1351 (1997)). Recombinant apyrase is commercially available from New England Biolabs. In some embodiments, the apyrase used in the compositions and methods disclosed herein is not derived from potato. In other embodiments, the apyrase used in the compositions and methods disclosed herein is not derived from a mammal.

“Mucosa” is a mucus-secreting membrane lining all body cavities or passages that communicate with the exterior. Mucosa is a moist tissue that lines many organs (such as the intestines) and body cavities (such as nose, mouth, lungs, vagina, bile duct, esophagus) and secretes mucous (a thick fluid). The mucosa, or mucous membrane, is a type of tissue protects body cavities from environmental conditions, pathogens and toxic substances and are usually moist tissues that are bathed by secretions (such as secretions in the bowel, lung, nose, mouth and vagina).

As used herein, “microorganism” or “microbe” refers to a bacterium, a fungus, a virus, a protozoan, and other microbes or microscopic organisms.

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide.

As used herein, “prevent,” “preventing,” “prevention” and grammatical variations thereof refers to a method of partially or completely delaying or precluding the onset or recurrence of a disorder or condition and/or one or more of its attendant symptoms or barring a subject from acquiring or reacquiring a disorder or condition or reducing an subject's risk of acquiring or reacquiring a disorder or condition or one or more of its attendant symptoms.

As used herein, the term “reducing” “or “decreasing” in relation to a particular trait, characteristic, feature, biological process, or phenomena refers to a decrease in the particular trait, characteristic, feature, biological process, or phenomena. The trait, characteristic, feature, biological process, or phenomena can be decreased by 5%, 10%, 15%, 20%, 25%, 30%, 35%, 40%, 45%, 50%, 55%, 60%, 65%, 70%, 75%, 80%, 85%, 90%, 95%, 100% or greater than 100%.

As used herein, the term “subject” or “patient” is meant a mammal (e.g., a human). In some embodiments, a subject is suffering from a relevant disease, disorder or condition including, but not limited to, antibiotic-associated diarrhea (AAD), Clostridium difficile-associated disease (CDAD), acquired immunodeficiency syndrome (AIDS), hypothyroidism, food poisoning, obesity, inflammatory bowel disease (IBD), irritable bowel syndrome (IBS). In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein “administer” or “administering” is meant the action of introducing one or more microbial strain, an exogenous feed enzyme and/or a strain and an exogenous feed enzyme to a subject, such as by feeding or by gavage.

As used herein, “effective amount” or “therapeutically effective amount” means a quantity of exogenous enzyme(s)s to improve one or more metrics in a subject. Improvement in one or more metrics of a subject (such as, without limitation, any of increased bodyweight gain, intestinal health status, improved gut barrier integrity, reduced mortality, or reduced pathogen infection) can be measured as described herein or by other methods known in the art. The exogenous enzymes can also be administered in one or more doses.

The term “intestinal health status” refers to the status of the gut wall structure and morphology which can be affected by, for example, infectious agents or a non-infectious cause, such as a suboptimal diet. “Gut wall structure and morphology” or “gut barrier integrity” can refer to, without limitation, epithelial damage and epithelial permeability which is characterized by a shortening of villi, a lengthening of crypts and an infiltration of inflammatory cells (such as, without limitation, CD3+ cells). The latter damage and inflammation markers can also be associated with a “severe” macroscopic appearance of the gut—compared to a “normal” appearance—when evaluated using a scoring system such as the one described by Teirlynck et al. (2011).

The term “commensal bacteria” refers to microflora (normal microflora, indigenous microbiota) consisting of those micro-organisms which are present on body surfaces covered by epithelial cells and are exposed to the external environment (gastrointestinal and respiratory tract, vagina, skin, etc.). These bacteria have been shown to impact the regulation of immunophysiological functions, including but not limited to, metabolism, ontogeny, and pathogen defense. Commensal bacteria have also been shown to promote resistance to gut pathogens that is mutually beneficial to the host and the commensal microbiota (Kahn et al, 2019, Frontiers in Immunology, 10:1203; Hogenova et al., 2004, Immune. Let., 93(2-3):97-108, incorporated by reference herein for discussion of commensal bacterial species).

The term “probiotic” refers to live or viable microorganisms which, when administered in effective amounts to a subject, confer a health benefit on the subject. For example, the probiotics included in the probiotic formulations described herein, which are suitable for oral or rectal administration to subjects, may improve the subjects' gastrointestinal or gut health. The term “probiotic,” as used herein, encompasses live microorganisms as well as viable microorganisms in dormant states, including frozen microorganisms, desiccated or dried microorganisms, spores, cysts or microorganisms in various states of reduced metabolic activity, which can be reconstituted upon exposure to suitable conditions. In some embodiments, the microorganisms of the probiotic comprise or consist of one or more commensal bacteria. In other embodiments, probiotic the microorganisms of the probiotic are derived from a fecal microbial source (such as fecal microbial source obtained from an otherwise healthy subject). Probiotics are distinguished from bacterial compositions that have been killed, for example, by pasteurization or heat treatment. Administration of non-viable bacterial compositions is also contemplated in certain embodiments of the methods disclosed herein.

As used herein, the term “engraftment” refers to the colonization of one or more bacterial species (such as commensal bacterial species provided, for example, as a probiotic) within the gut or to the adherence of one or more bacterial species (such as commensal bacterial species provided, for example, as a probiotic or as a fecal transplant) onto the mucosal surface of gut epithelial cells in a subject.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number.

For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Compositions

### A. Enzymes

Provided herein are isolated proteins having phosphatase activity. A phosphatase is an enzyme that dephosphorylates its substrates; i.e., it hydrolyses phosphoric acid monoesters into a phosphate ion and a molecule with a free hydroxyl group. Phosphatases include apyrases. This action is directly opposite to that of phosphorylases and kinases, which attach phosphate groups to their substrates by using energetic molecules like ATP.

The terms “protein” and “polypeptide” refer to compounds comprising amino acids joined via peptide bonds and may be used interchangeably. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Amino acid positions in a given polypeptide sequence can be named by the one letter code for the amino acid, followed by a position number. For example, a glycine (G) at position 87 is represented as “G087” or “G87.”

As used herein, where “amino acid sequence” is recited it refers to an amino acid sequence of a protein or peptide molecule. An “amino acid sequence” can be deduced from the nucleic acid sequence encoding the protein. However, terms such as “polypeptide” or “protein” are not meant to limit the amino acid sequence to the deduced amino acid sequence but can include posttranslational modifications of the deduced amino acid sequences, such as amino acid deletions, additions, and modifications such as glycosylations and addition of lipid moieties. Also, the use of non-natural amino acids, such as D-amino acids to improve stability or pharmacokinetic behavior falls within the scope of the term “amino acid sequence”, unless indicated otherwise.

The terms “signal sequence” and “signal peptide” refer to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “mature” form of a protein, polypeptide, or peptide refers to the functional form of the protein, polypeptide, or enzyme without the signal peptide sequence and propeptide sequence.

The term “wild-type” in reference to an amino acid sequence or nucleic acid sequence indicates that the amino acid sequence or nucleic acid sequence is a native or naturally-occurring sequence. As used herein, the term “naturally-occurring” refers to anything (e.g, proteins, amino acids, or nucleic acid sequences) that is found in nature. Conversely, the term “non-naturally occurring” refers to anything that is not found in nature (e.g, recombinant/engineered nucleic acids and protein sequences produced in the laboratory or modification of the wild-type sequence).

The phosphatases for use in the compositions and methods disclosed herein are active at least from about pH 3 to pH 7 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7) or from pH 3.5 to 5.5 or from pH 3.5 to 7 or from pH 5 to 7. The enzyme can be a member of the GDA1 CD39 superfamily (excluding apyrases derived from potato) or an acid phosphatase. In some embodiments, the enzyme comprises an amino acid sequence having at least 40% or at least 60% sequence identity (such as any of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) with the full length amino acid sequence of SEQ ID NO: 1 or SEQ ID NO:9 (CRC22110). In other embodiments, the enzyme comprises an amino acid sequence having at least 50% or at least 66% sequence identity (such as any of about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) with the full length amino acid sequence of SEQ ID NO: 2 or SEQ ID NO:18 (CRC21323).

Other phosphatases for use in the compositions and methods disclosed herein are active at least from about pH 3 to pH 7 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7) or from pH 3.5 to 5.5 or from pH 3.5 to 7 or from pH 5 to 7 and are a member of the GDA1 CD39 superfamily (excluding apyrases derived from potato). In some embodiments, the enzyme comprises an amino acid sequence having at least 40% or at least 60% sequence identity (such as any of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) with the full length amino acid sequence of one or more of SEQ ID NO: 6, SEQ ID NO: 7, SEQ ID NO: 8, SEQ ID NO: 10, SEQ ID NO: 11, SEQ ID NO: 12, SEQ ID NO: 13, SEQ ID NO: 14, SEQ ID NO: 15, or SEQ ID NO: 16.

In further embodiments, phosphatases for use in the compositions and methods disclosed herein 1) are active at least from about pH 3 to pH 7 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7) or from pH 3.5 to 5.5 or from pH 3.5 to 7 or from pH 5 to 7; 2) comprises an amino acid sequence having at least 40% or at least 60% sequence identity (such as any of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) with the full length amino acid sequence of one or more of SEQ ID NO: 6, SEQ ID NO: 7, SEQ ID NO: 8, SEQ ID NO:9, SEQ ID NO: 10, SEQ ID NO: 11, SEQ ID NO: 12, SEQ ID NO: 13, SEQ ID NO: 14, SEQ ID NO: 15, or SEQ ID NO: 16; and 3) have amino acid residues S204, F205, L206, G207, L208, and G209 at positions corresponding to the numbering of SEQ ID NO:9.

Yet additional phosphatases for use in the compositions and methods disclosed herein are active at least from about pH 3 to pH 7 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7) or from pH 3.5 to 5.5 or from pH 3.5 to 7 or from pH 5 to 7 and are a member of the acid phosphatase (Class A) family. In other embodiments, the enzyme comprises an amino acid sequence having at least 50% or at least 66% sequence identity (such as any of about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) with the full length amino acid sequence of SEQ ID NO:17, SEQ ID NO: 18, SEQ ID NO: 19, SEQ ID NO: 20, SEQ ID NO: 21, SEQ ID NO: 22, SEQ ID NO: 23, or SEQ ID NO: 24.

### B. Vectors

The terms “nucleic acid,” “ polynucleotide”, and “nucleic acid fragment” are used interchangeably herein and refer to a polymer of RNA or DNA that is single- or double- stranded, optionally containing synthetic, non-natural or altered nucleotide bases. An isolated nucleic acid molecule in the form of a polymer of DNA may be comprised of one or more segments of cDNA, genomic DNA or synthetic DNA.

A DNA construct comprising a nucleic acid encoding an acid phosphatase polypeptide disclosed herein (such as a nucleic acid comprising any of SEQ ID NOs:25-43) can be constructed such that it is suitable to be expressed in a host cell. Because of the known degeneracy in the genetic code, different polynucleotides that encode an identical amino acid sequence can be designed and made with routine skill. It is also known that, depending on the desired host cells, codon optimization may be required prior to attempting expression.

A polynucleotide encoding an acid phosphatase polypeptide of the present disclosure can be incorporated into a vector. Vectors can be transferred to a host cell using known transformation techniques, such as those disclosed below.

A suitable vector may be one that can be transformed into and/or replicated within a host cell. For example, a vector comprising a nucleic acid encoding an acid phosphatase polypeptide disclosed herein can be transformed and/or replicated in a bacterial host, fungal, or mammalian cell as a means of propagating and amplifying the vector. The vector may also be suitably transformed into an expression host, such that the encoding polynucleotide is expressed as a functional acid phosphatase enzyme.

A non-limiting representative useful vector is pTrex3gM (see, Published US Patent Application 20130323798), pTTT (see, Published US Patent Application 20110020899), and p2JM103BBI (see Vogtentanz, Protein Expr Purif, 55:40-52, 2007), which can be inserted into genome of host. The vectors pTrex3gM, pTTT, and p2JM103BBI can be modified with routine skill such that they comprise and express a polynucleotide encoding an acid phosphatase polypeptide of the invention.

An expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the acid phosphatase polypeptide to a host cell organelle such as a peroxisome, or to a particular host cell compartment. For expression under the direction of control sequences, the nucleic acid sequence of the acid phosphatase polypeptide is operably linked to the control sequences in proper manner with respect to expression.

A polynucleotide encoding an acid phosphatase polypeptide disclosed herein can be operably linked to a promoter, which allows transcription in the host cell. The promoter may be any DNA sequence that shows transcriptional activity in the host cell of choice and may be derived from genes encoding proteins either homologous or heterologous to the host cell. Examples of promoters for directing the transcription of the DNA sequence encoding an acid phosphatase, for example in a bacterial, fungal, or mammalian host, include the aprE promoter (SEQ ID NO. 3)), the promoter of the lac operon of E. coli, the Streptomyces coelicolor agarase gene dagA or celA promoters, the promoters of the Bacillus licheniformis amylase gene (amyL), the promoters of the Bacillus stearothermophilus maltogenic amylase gene (amyM), the promoters of the Bacillus amyloliquefaciens amylase (amyQ), the promoters of the Bacillus subtilis xylA and xylB genes, and the like.

For transcription in a fungal host, examples of useful promoters include those derived from the gene encoding Aspergillus oryzae TAKA amylase, Rhizomucor miehei aspartic proteinase, Aspergillus niger neutral α-amylase, Aspergillus niger acid stable α-amylase, Aspergillus niger glucoamylase, Rhizomucor miehei lipase, Aspergillus oryzae alkaline protease, Aspergillus oryzae triose phosphate isomerase, Aspergillus nidulans acetamidase and the like. When a gene encoding an acid phosphatase polypeptide is expressed in a bacterial species such as an E. coli, a suitable promoter can be selected, for example, from a bacteriophage promoter including a T7 promoter and a phage lambda promoter. Along these lines, examples of suitable promoters for the expression in a yeast species include but are not limited to the Gal 1 and Gal 10 promoters of Saccharomyces cerevisiae and the Pichia pastoris AOX1 or AOX2 promoters. Expression in filamentous fungal host cells often involves cbh1, which is an endogenous, inducible promoter from T. reesei. See Liu et al. (2008) Acta Biochim. Biophys. Sin (Shanghai) 40(2): 158-65.

The coding sequence can be operably linked to a signal sequence. The DNA encoding the signal sequence may be a DNA sequence naturally associated with the acid phosphatase polypeptide gene of interest to be expressed or may be from a different genus or species from which a portion of the acid phosphatase is derived. A signal sequence and a promoter sequence comprising a DNA construct or vector can be introduced into a fungal host cell and can be derived from the same source. For example, the signal sequence may be the Trichoderma reesei cbh1 signal sequence, which is operably linked to a cbh1 promoter.

An expression vector may also comprise a suitable transcription terminator and, in eukaryotes, polyadenylation sequences operably linked to the DNA sequence encoding an acid phosphatase. Termination and polyadenylation sequences may suitably be derived from the same sources as the promoter.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., Published International PCT Application WO 91/17243.

Synthetic genes coding for the protein sequences corresponding to SEQ ID NOs. 1, 2, 6, 7, 8, 9, 10, 11, 12, 13, 14, 15, 16, 17, 18, 19, 20, 21, 22, 23, or 24 can be generated using molecular biology methods known in the art. These genes can further be cloned into a suitable expression vector resulting in expression plasmids containing one or more of: a promoter (for example, the aprE promoter (SEQ ID NO. 3)); a signal sequence (for example, the aprE signal sequence (SEQ ID NO. 4); an oligonucleotide to facilitate the secretion of the target protein (for example, an oligonucleotide encoding the peptide Ala-Gly-Lys); a synthetic nucleotide sequence encoding the mature region of the gene of interest (for example, a synthetic oligonucleotide having at least 50% or at least 66% sequence identity (such as any of about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) with the synthetic oligonucleotide of any of SEQ ID NOs:25, 26, 27, 28, 29, 30, 31, 32, 33, 34, 35, 36, 37, 38, 39, 40, 41, 42, or 43); and/or a terminator (such as the AprE terminator (SEQ ID NO. 5)). The expression plasmid can further be transformed into a suitable expression host cell (such as a mammalian or bacterial or fungal expression host cell).

### C. Host Cells

An isolated cell, either comprising a DNA construct (such as any of the DNA constructs disclosed herein) or an expression vector (such as any of the expression vectors disclosed herein, for example, an expression vector comprising any of the polynucleotides of SEQ ID NOs:25-43 that encodes a polypeptide of any one of SEQ ID NOs:1-2 or 6-24), is advantageously used as a host cell in the recombinant production of an acid phosphatase polypeptide. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector in connection with the different types of host cells.

Examples of suitable bacterial host cells are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host cell can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species.

Suitable host cells among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. An acid phosphatase polypeptide expressed by a fungal host cell can be glycosylated, i.e., will comprise a glycosyl moiety. The glycosylation pattern can be the same or different as present in the wild-type acid phosphatase. The type and/or degree of glycosylation may impart changes in enzymatic and/or biochemical properties.

Suitable mammalian host cells include, without limitation, Chinese hamster ovary (CHO) cells or human embryonic kidney HEK) cells. Additional host cells can include insect sells, such as S2 cells.

It is advantageous to delete genes from expression hosts, where the gene deficiency can be cured by the transformed expression vector. Known methods may be used to obtain a fungal host cell having one or more inactivated genes. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding an acid phosphatase is stably integrated into a host cell chromosome. Transformants are then selected and purified by known techniques.

A method of producing any of the acid phosphatase polypeptides disclosed herein may comprise cultivating a host cell under conditions conducive to the production of the enzyme and recovering the enzyme from the cells and/or culture medium.

The medium used to cultivate the cells may be any conventional medium suitable for growing the host cell and obtaining expression of an acid phosphatase polypeptide. Suitable media and media components are available from commercial suppliers or may be prepared according to published recipes (e.g., as described in catalogues of the American Type Culture Collection).

Any of the fermentation methods well known in the art can suitably be used to ferment the transformed or the derivative fungal strain as described above. In some embodiments, fungal cells are grown under batch or continuous fermentation conditions.

Separation and concentration techniques are known in the art and conventional methods can be used to prepare a concentrated solution or broth comprising an acid phosphatase polypeptide of the invention.

After fermentation, a fermentation broth is obtained, the microbial cells and various suspended solids, including residual raw fermentation materials, are removed by conventional separation techniques in order to obtain an acid phosphatase solution. Filtration, centrifugation, microfiltration, rotary vacuum drum filtration, ultrafiltration, centrifugation followed by ultra-filtration, extraction, or chromatography, or the like, are generally used.

It may at times be desirable to concentrate a solution or broth an acid phosphatase polypeptide to optimize recovery. Use of un-concentrated solutions or broth would typically increase incubation time in order to collect the enriched or purified enzyme precipitate.

### D. Enzyme Compositions

Any of the enzymes for use in the methods disclosed herein can be formulated in compositions (for example, pharmaceutical or nutritional compositions). As already mentioned, a herein described protein having phosphatase activity at acid pH ranges according to the invention is useful in treating and/or preventing diseases associated with inflammation in the gut. In one embodiment, the invention provides a composition, preferably a pharmaceutical composition, comprising a protein according to the invention. Said pharmaceutical composition optionally comprises a pharmaceutical acceptable carrier, diluent or excipient.

The composition can be presented in any form, for example as a tablet, as an injectable fluid or as an infusion fluid etc. Moreover, the composition, protein, nucleotide and/or vector according to the invention can be administered via different routes, for example intravenously, rectally, bronchially, or orally. Yet another suitable route of administration is the use of a duodenal drip.

In a one embodiment, the used route of administration is the intravenous route. It is clear for the skilled person, that preferably an effective amount of a protein according to the invention is delivered. As a start point, 1-50,000 U/kg/day can be used. Another suitable route, e.g., for HPP, is the subcutaneous route. If the intravenous route of administration is used, a protein according to the invention can be (at least for a certain amount of time) applied via continuous infusion.

Said composition according to the invention can optionally comprise pharmaceutically acceptable excipients, stabilizers, activators, carriers, permeators, propellants, disinfectants, diluents and preservatives. Suitable excipients are commonly known in the art of pharmaceutical formulation and may be readily found and applied by the skilled artisan, references for instance Remmington's Pharmaceutical Sciences, Mace Publishing Company, Philadelphia Pa., 17th ed. 1985.

For oral administration, the protein can, for example, be administered in solid dosage forms, such as capsules, tablets (e.g., with an enteric coating), and powders, or in liquid dosage forms, such as elixirs, syrups, and suspensions. Acid phosphatases and/or apyrases can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable colour, taste, stability, buffering capacity, dispersion or other known desirable features are red iron oxide, silica gel, sodium lauryl sulphate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Enteric coatings arrest the release of the active compound from orally ingestible dosage forms. Depending upon the composition and/or thickness, the enteric coatings are resistant to stomach acid for required periods of time before they begin to disintegrate and permit slow release of acid phosphatase and/or apyrase in the lower stomach, small intestines, or large intestine. Examples of some enteric coatings are disclosed in U.S. Pat. No. 5,225,202 (incorporated by reference). Examples of enteric coatings comprise beeswax and glyceryl monostearate; beeswax, shellac and cellulose, optionally with neutral copolymer of polymethacrylicacid esters; copolymers of methacrylic acid and methacrylic acid methylesters or neutral copolymer of polymethacrylic acid esters containing metallic stearates (for references enteric coatings see: U.S. Pat. Nos. 4,728,512, 4,794,001, 3,835,221, 2,809,918, 5,225,202, 5,026,560, 4,524,060, 5,536,507). Most enteric coating polymers begin to become soluble at pH 5.5 and above, with a maximum solubility rates at pH above 6.5. Enteric coatings may also comprise subcoating and outer coating steps, for instance for pharmaceutical compositions intended for specific delivery in the lower GI tract, i.e. in the colon (pH 6.4 to 7.0, ileum pH 6.6), as opposed to a pH in the upper intestines, in the duodenum of the small intestines the pH ranges 7.7-8 (after pancreatic juices and bile addition). The pH differences in the intestines may be exploited to target the enteric-coated acid phosphatase and/or apyrase composition to a specific area in the gut. It also allows the selection of a specific acid phosphatase and/or apyrase enzyme that is most active at a particular pH in the intestine.

Besides the fact that a protein according to the invention can be incorporated in a pharmaceutical composition such an acid phosphatase and/or apyrase can also be part of a nutritional composition or a nutraceutical.

A protein according to the invention can be added to a nutrient (such as milk) but can also be produced within said nutrient (for instance by molecular engineering). Moreover, tablets and/or capsules can be prepared which are subsequently added to a nutrient or which can be taken directly by a human being.

In yet another aspect, the invention features beverage and food products comprising an acid phosphatase and/or an apyrase effective to treat or prevent gut inflammation in a subject in need thereof. The beverage products can contain from 1 to 10,000, e.g., 1 to 200, 200 to 500, 500 to 1,000, 1,000 to 5,000, or 5,000 to 10,000, units per milliliter. The food products can contain from 1 to 10,000, e.g., 1 to 200, 200 to 500, 500 to 1,000, 1,000 to 5,000, or 5,000 to 10,000, units per gram.

### III. Methods

### A. Methods for Treating a Disorder Characterized by Gut Inflammation

Provided herein are methods and compositions for decreasing or preventing a disorder characterized by inflammation in the gut in a subject in need thereof. Without being bound to theory, it is believed that administration of acid phosphatases and/or apyrases (such as an acid phosphatase and/or apyrase at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of any of SEQ ID NO:1, SEQ ID NO:6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16 or at least about 50%, at least about 55%, at least about 60%, at least about 65%, at least about 66%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of SEQ ID NO:2, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24) can alleviate inflammation by removing gut-microbe generated sources of inflammation by, for example, detoxification of LPS in situ at mucosal tissues in body cavities and/or removal of exATP, CpG DNA, and flagellin.

One non-limiting aim of in situ detoxification of LPS at mucosal surfaces and/or removal of exATP, CpG DNA, and flagellin in the body is to prevent or reduce local inflammatory response at such surfaces. Furthermore, again without being bound to theory, the LPS, exATP, CpG DNA, and flagellin that is thus detoxified is no longer available for passage through mucosal layers and thus cannot enter the circulation where it will exert its toxic effects and/or cause a further local and/or systemic inflammatory response. The methods comprise the use of sources of acid phosphatase and/or apyrase.

A source of acid phosphatase and/or apyrase can be any acid phosphatase and/or apyrase enzyme, or any composition comprising the acid phosphatase and/or apyrase enzyme and any means which is capable of producing a functional acid phosphatase and/or apyrase enzyme in the context of the current invention, such as DNA or RNA nucleic acids encoding an acid phosphatase and/or apyrase enzyme. The nucleic acid encoding acid phosphatase and/or apyrase may be embedded in suitable vectors such as plasmids, phagemids, phages, (retro)viruses, transposons, gene therapy vectors and other vectors capable of inducing or conferring production of acid phosphatase and/or apyrase. Also, native or recombinant microorganisms, such as bacteria, fungi, protozoa and yeast may be applied as a source of acid phosphatase and/or apyrase in the context of the current invention.

In one embodiment, the invention provides a method for the prevention or reduction of gut inflammation (such as inflammation mediated or caused by LPS toxicity at a mucosal lining of a mammalian body cavity or the presence of exATP, CpG DNA, and flagellin) comprising the step of administering a source of acid phosphatase and/or apyrase (such as an acid phosphatase and/or apyrase at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of any of SEQ ID NO:1, SEQ ID NO:6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16 or at least about 50%, at least about 55%, at least about 60%, at least about 65%, at least about 66%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of SEQ ID NO:2, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24) to the mucosal layer and/or the gut. For those jurisdictions where methods of treatment are unpatentable by law, the invention likewise pertains to the use of acid phosphatase and/or apyrase as defined above, or the use of a composition containing a source of acid phosphatase and/or apyrase as defined above. The source of acid phosphatase and/or apyrase is used for the manufacture of a medicament for delivery of acid phosphatase and/or apyrase at a mucosal layer and/or the gut for the prevention or reduction of inflammation of gut tissues.

In particular the above-mentioned method of administering a source of acid phosphatase and/or apyrase at mucosal layers of body cavities (such as the gut, for example the large and/or small intestine) is suited for the treatment or prophylaxis of gut inflammation (for example, inflammation caused by LPS toxicity or the presence of exATP, CpG DNA, and/or flagellin brought about by dysbiosis of the gut microbiome) and associated diseases, although the method may also be advantageously used for healthy subjects as a prophylactic treatment aimed at the prevention of gut inflammation (for example, inflammation caused by LPS toxicity or the presence of exATP, CpG DNA, and/or flagellin brought about by dysbiosis of the gut microbiome) and associated diseases. The beneficial effects of acid phosphatase and/or apyrase administration to reduce gut inflammation and at mucosal layers according to the current invention will generate a general health promoting effect regardless of the medical condition of the subject treated. The health promoting effect may be further augmented by the consequent decrease in LPS, exATP, CpG DNA, and/or flagellin influx through mucosal layers.

An LPS, exATP, CpG DNA, and/or flagellin mediated or induced disease may be any disease, symptom or group of symptoms caused by LPS, exATP, CpG DNA, and/or flagellin toxicity. An LPS, exATP, CpG DNA, and/or flagellin exacerbated disease may be any disease or symptom that is not directly caused by LPS, exATP, CpG DNA, and/or flagellin in the gut but a disease which symptoms and clinical features may be aggravated by LPS, exATP, CpG DNA, and/or flagellin and the clinical state of the subject suffering from such a disease is worsened by the presence LPS, exATP, CpG DNA, and/or flagellin in the gut.

Preferably the method is aimed at the treatment of an LPS, exATP, CpG DNA, and/or flagellin-mediated or exacerbated diseases such as inflammatory bowel diseases, in particular in patients diagnosed with Crohn's disease and ulcerative colitis. Its presence is the consequence of the damaged intestinal mucosa and increased LPS, exATP, CpG DNA, and/or flagellin influx or gut translocation and causes or exacerbates the inflammatory response in the intestines. Intestinal bacterial translocation and LPS gut translocation is also observed in acute pancreatitis and liver diseases caused by cirrhosis, alcohol abuse, obstructive jaundice and other hepatic conditions. Endotoxin has also been implicated in the development of periodontal disease, where it penetrates the gingival epithelium/mucosa, ensuing a local inflammatory response. In a preferred embodiment the method comprises oral administration of a source of acid phosphatase and/or apyrase to reduce LPS, exATP, CpG DNA, and/or flagellin toxicity at and/or passage of LPS, exATP, CpG DNA, and/or flagellin through the mucosa.

In one non-limiting embodiment, the mode of administration comprises the use of pharmaceutical compositions comprising sources of acid phosphatase and/or apyrase, which may be delivered in a daily doses regimen to reduce gut inflammation in the lumen of the GI tract for a prolonged period of time. Preferably the pharmaceutical compositions comprise an enteric coating to protect acid phosphatase and/or apyrase from the detrimental effects of gastric juices (pH 1.0 to 2.5) and ensure efficient delivery of acid phosphatase and/or apyrase at the mucosa of the intestinal tract. In another embodiment, the pharmaceutical composition is a source of acid phosphatase and/or apyrase comprised within an enteric coat.

Mucosal tissues to be treated according to the current invention are the mucosal tissues lining the intestinal tract body cavities. Orally administered acid phosphatase and/or apyrase is delivered at the mucosal tissues of the GI tract, which comprises the esophagus, stomach, the small intestines or bowel, (duodenum, jejunum, ileum) and large intestines or colon (caecum, ascending colon, transverse colon, descending colon, sigmoid colon, rectum and anus). Within the scope of the current invention, also mucosal tissues lining the mouth, the ducts of the bile and the pancreas are part of the intestinal tract and may be treated according to method of the current invention.

The compositions comprising a source of acid phosphatase and/or apyrase according to the current invention are particularly suited for oral administration to prevent treat, reduce, treat or alleviate inflammatory diseases of the gastrointestinal tract. Inflammatory diseases of the gastrointestinal tract may be induced and/or exacerbated significantly by the influx of bacterially derived substances such as LPS, exATP, CpG DNA, and/or flagellin. For example, and without being bound to theory, a reduction in the amount of toxic LPS in the lumen of the intestines by administration of sources of acid phosphatase and/or apyrase will, through detoxification of the lipid A moiety of LPS, result in a corresponding decrease in the systemic influx of toxic LPS in the circulation of a subject. In one embodiment, the oral administration of sources of acid phosphatase and/or apyrase are particularly preferred for the prophylaxis or treatment of the following inflammatory disease of the gastrointestinal tract: Crohn's disease, colitis, (necrotizing) enterocolitis, colitis ulcerosa, hepatobiliary disease, hepatitis B, hepatitis C, liver cirrhosis, liver fibrosis, bile duct inflammation, biliary obstruction, pancreatitis, acute pancreatitis, peritonitis and periodontal disease.

In another embodiment of the invention, a source of acid phosphatase and/or apyrase is orally administered to subjects who suffer from an increased mucosal permeability of the gastrointestinal tract. Increased mucosal permeability of the GI tract is often the result of a decreased perfusion or ischemia of the intestines. Ischemia, a lack of oxygen supply by the bloodstream, may be caused by heart failure, injuries, trauma or surgery. Without being bound to theory, ischemia of the intestines results in a malfunctioning of the mucosa and a consequential increase in the influx or translocation of toxic LPS (and other sources of inflammation such as exATP, CpG DNA, and/or flagellin) from the gut, resulting in both local and systemic toxicity and inflammation. The toxicity and inflammatory response may even further enhance the mucosal permeability, resulting in a vicious circle. Increased mucosal permeability of the GI tract may be the result of inflammatory bowel diseases or other pathological conditions of the GI tract. Oral administration of sources of acid phosphatase and/or apyrase according to the current invention will significantly reduce or abolish this increased influx off inflammatory agents such as LPS and/or exATP, CpG DNA, and/or flagellin in the lumen of the intestinal cavities.

The current invention also provides compositions comprising a source of acid phosphatase and/or apyrase, amongst which are pharmaceutical and nutraceutical compositions comprising a source of acid phosphatase and/or apyrase. The compositions may optionally comprise pharmaceutically acceptable excipients, stabilizers, activators, carriers, permeators, propellants, disinfectants, diluents and preservatives. Suitable excipients are commonly known in the art of pharmaceutical formulation and may be readily found and applied by the skilled artisan, references for instance Remmington's Pharmaceutical Sciences, Mace Publishing Company, Philadelphia Pa., 17th ed. 1985. In a preferred embodiment the compositions comprising a source of acid phosphatase and/or apyrase are suitable for oral administration and comprise an enteric coating to protect the acid phosphatase and/or apyrase from the adverse effects of gastric juices and low pH. Enteric coating and controlled release formulations are well known in the art (references as described above). Enteric coating compositions in the art may comprise of a solution of a water-soluble enteric coating polymer mixed with the active ingredient(s) such as acid phosphatase and/or apyrase and other excipients, which are dispersed in an aqueous solution and which may subsequently be dried and/or pelleted. The enteric coating formed offers resistance to attack of acid phosphatase and/or apyrase by atmospheric moisture and oxygen during storage and by gastric fluids and low pH after ingestion, while being readily broken down under the alkaline conditions which exist in the lower intestinal tract.

Acid phosphatase and/or apyrase containing compositions for the delivery of acid phosphatase and/or apyrase at mucosal tissues for treatment and prevention of gut inflammation according to the current invention can comprise a microbially-derived (i.e. from bacteria, archaea, fungi, or yeast) acid phosphatase and/or apyrase eukaryotic acid phosphatase and/or apyrase.

### B. Methods for Promoting Engraftment of Commensal Bacteria in the Gut

Also provided herein is a method for promoting the engraftment of one or more commensal bacteria into the gut of a subject. The method requires at least the administering to the subject any of the acid phosphatase and/or apyrase compositions disclosed herein (such as an acid phosphatase and/or apyrase at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of any of SEQ ID NO:1, SEQ ID NO:6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16 or at least about 50%, at least about 55%, at least about 60%, at least about 65%, at least about 66%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% identical to the amino acid sequence of SEQ ID NO:2, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24) and a source of one or more commensal bacteria. In some embodiments, the method promotes engraftment of the one or more commensal bacteria into the gut of a subject to a greater extent (such as about a 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, or more, inclusive of percentages falling in between these values, greater extent) compared to when the source of one or more commensal bacteria is administered in the absence of the acid phosphatase and/or apyrase compositions disclosed herein. Non-limiting examples of commensal bacteria include Ligilactobacillus ruminis, Prevotella copri, Ligilactobacillus salivarius, and Bifidobacterium longum. The source of one or more commensal bacteria can be administered as a probiotic (for example, orally or rectally) or as a fecal microbial transplant (for example, rectally or orally in a capsule).

Generally, the source of one or more commensal bacteria (such as probiotic compositions) comprise bacteria, such as one or more bacterial strains. In some embodiments of the invention, the source of one or more commensal bacteria is formulated in freeze-dried or lyophilized form. For example, the source of one or more commensal bacteria can comprise granules or gelatin capsules, for example hard gelatin capsules, comprising a bacterial strain disclosed herein.

In some embodiments, the source of one or more commensal bacteria comprise lyophilized bacteria. Lyophilization of bacteria is a well-established procedure in the art. Alternatively, the microbial-containing compositions can comprise a live, active bacterial culture.

In some embodiments, the source of one or more commensal bacteria is encapsulated to enable delivery of the bacterial strain to the intestine. Encapsulation protects the composition from degradation until delivery at the target location through, for example, rapturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule.

The source of one or more commensal bacteria can be administered orally and may be in the form of a tablet, capsule or powder. Other ingredients (such as vitamin C or minerals, for example), may be included as oxygen scavengers and prebiotic substrates to improve the delivery and/or partial or total colonization and/or engraftment and/or survival in vivo. Alternatively, the source of one or more commensal bacteria (such as probiotic compositions) can be administered orally as a food or nutritional product, such as milk or whey based fermented dairy product, or as a pharmaceutical product.

The source of one or more commensal bacteria can be formulated as a probiotic. Alternatively, the source of one or more commensal bacteria can be formulated as a non-viable bacterial compositions, such as a pasteurized or heat-treated bacterial composition.

A source of one or more commensal bacteria includes a therapeutically effective amount of a bacterial strain disclosed herein (for example, without limitation, one or more of Ligilactobacillus ruminis, Prevotella copri, Ligilactobacillus salivarius, and Bifidobacterium longum). A therapeutically effective amount of a bacterial strain is sufficient to exert a beneficial effect upon a patient. A therapeutically effective amount of a source of one or more commensal bacteria may be sufficient to result in delivery to and/or partial or total engraftment and/or colonization of the subject's intestine.

A suitable daily dose of the commensal bacteria, for example for an adult human, may be from about 1×10³ to about 1×10¹¹ colony forming units (CPU); for example, from about 1×10⁷ to about 1×10¹⁰ CPU; in another example from about 1×10⁶ to about 1×10¹⁰ GPU; in another example from about 1×10⁷ to about 1×10¹¹ CPU; in another example from about 1×10⁸ to about 1×10¹⁰ CPU; in another example from about 1×10⁸ to about 1×10¹¹ CPU. In certain embodiments, the dose of the bacteria is at least 10 9 cells per day, such as at least 10¹⁰, at least 10¹¹ or at least 10¹² cells per day.

In certain embodiments, the source of one or more commensal bacteria contains the bacterial strain in an amount of from about 1×10⁶ to about 1×10¹¹ CFU/g, respect to the weight of the composition; for example, from about 1×10⁸ to about 1×10¹⁰ CFU/g. The dose may be, for example, 1 g, 3 g, 5 g, and 10 g. In certain embodiments, the amount of the bacterial strain is from about 1×10³ to about 1×10¹¹ colony forming units per gram with respect to a weight of the composition.

In certain embodiments, any of the sources of one or more commensal bacteria is administered at a dose of between 500 mg and 1000 mg, between 600 mg and 900 mg, between 700 mg and 800 mg, between 500mg and 750 mg or between 750 mg and 1000 mg. In certain embodiments, the lyophilized bacteria in any of the microbial-containing compositions disclosed herein is administered at a dose of between 500 mg and 100 mg, between 600 mg and 900 mg, between 700 mg and 800 mg, between 500 mg and 750 mg or between 750 mg and 1000 mg.

Typically, a probiotic, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract Known prebiotics include commercial products such as inulin and transgalacto-oligosaccharides.

In certain embodiments, a probiotic composition is formulated to include a prebiotic compound in an amount of from about 1 to about 30% by weight respect to the total weight composition, (e.g. from 5 to 20% by weight). Carbohydrates may be selected from the group consisting of: fructo-oligosaccharides (or FOS), short-chain fructo-oligosaccharides, inulin, isomaltoseoligosaccharides, pectins, xylo-oligosaccharides (or XOS), chitosan-oligosaccharides (or COS), human milk oligosaccharides, beta-glucans, gum arabic modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded. In another embodiment, the prebiotic can include one or more polyphenols (such as a plant polyphenol). In some embodiments, any of the probiotics disclosed herein can be formulated with additional probiotics derived from the genera Lactobacillus and Bifidobacterium (such as B. lactis B420).

The source of one or more commensal bacteria can further comprise pharmaceutically acceptable excipients or carriers. Acceptable carriers or diluents for therapeutic use are well known in the pharmaceutical art. Examples of suitable carriers include, without limitation, lactose, starch, glucose, methyl cellulose, magnesium stearate, mannitol, sorbitol and the like. Examples of suitable diluents include, without limitation, ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binders, lubricants, suspending agents, coating agents (such as a gastric-resistant enteric coating agent that does not dissolve or degrade until reaching the small or large intestine), or solubilizing agents. Examples of suitable binders include, without limitation, starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include, without limitation, sodium oleate, sodium stearate, magnesium stearate, sodium benzoate, sodium acetate, sodium chloride and the like. Preservatives, stabilizers, dyes and even flavoring agents may be provided in the pharmaceutical composition. Examples of preservatives include, without limitation, sodium benzoate, sorbic acid and esters of p-hydroxybenzoic acid. Antioxidants and suspending agents may be also used.

The source of one or more commensal bacteria can be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the microbial-containing composition is formulated as a milk-based product. The term “milk-based product,” as used herein, means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavored milks, ice cream; milk-containing food such as sweets.

In certain embodiments, the source of one or more commensal bacteria contain a single bacterial strain or species and do not contain any other bacterial strains or species. Such compositions may comprise only de minimis or biologically irrelevant amounts of other bacterial strains or species. Such compositions may be a culture that is substantially free from other species of organism. In certain embodiments, the compositions of the invention consist of 1, 2, 3, 4, 5, 6, 7, 8, 9, 10, 11, 12, 13, 14, 15 or 16 bacterial strains or species. In certain embodiments, the compositions consist of from 1 to 10, such as from 1 to 5 bacterial strains or species.

The source of one or more commensal bacteria for use in accordance with the methods disclosed herein may or may not require marketing approval.

In certain embodiments, the source of one or more commensal bacteria is stored in a sealed container at about 4° C. or about 25° C. and the container is placed in an atmosphere having 50% relative humidity, at least 80%, 70%, 60%, 50%, 40%, 30%, 20%, or 10% of the bacterial strain as measured in colony forming units, remains after a period of at least about 1 month, 3 months, 6 months, 1 year, 1.5 years, 2 years, 2.5 years or 3 years.

The invention can be further understood by reference to the following examples, which are provided by way of illustration and are not meant to be limiting.

### EXAMPLES

### Example 1: Characterization of Enzyme Candidates

Phylogenetically diverse enzymes were identified from the NCBI database as shown in Table 1. These enzymes were cloned, expressed, and purified for enzyme assays to determine their ability to hydrolyze ATP. Potato apyrase (A6535) and CD39 (SRP0623) were purchased from Sigma Aldrich.

TABLE 1
NCBI sequence identifiers for cloned enzymes

| Sequence name | NCBI identifier |
|---|---|
| CRC22110 | EKE69473.1 |
| CRC22105 | WP_045094910.1 |
| CRC22112 | KTD66016.1 |
| CRC22383 | WP_058475114.1 |
| CRC21328 | CCD05982.1 |
| CRC21323 | WP_054306375.1 |
| CRC21322 | AEG96708.1 |
| CRC21320 | QHO60207.1 |

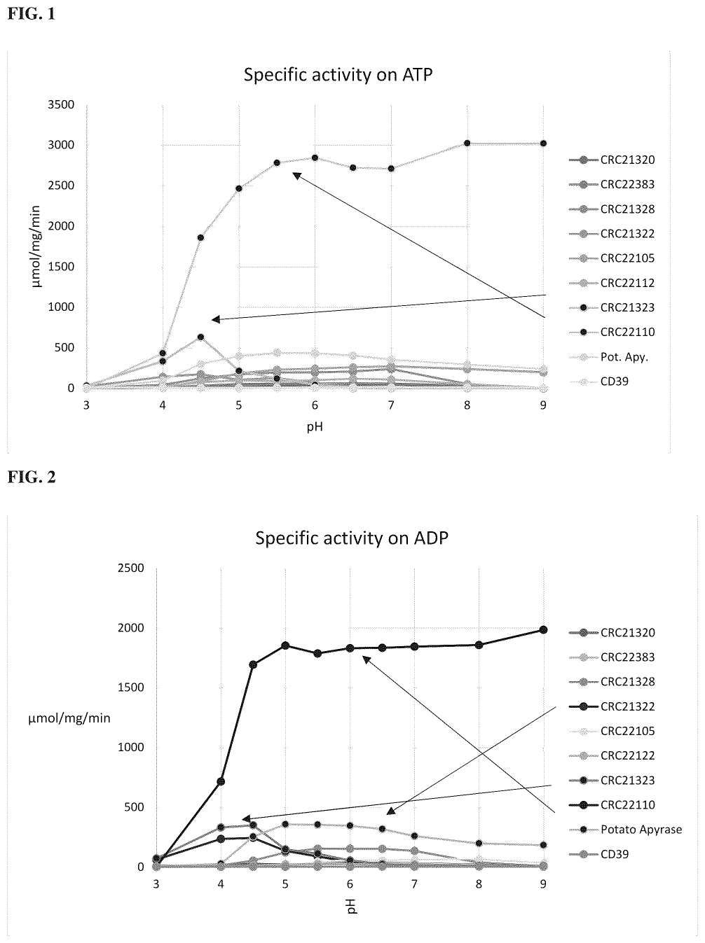

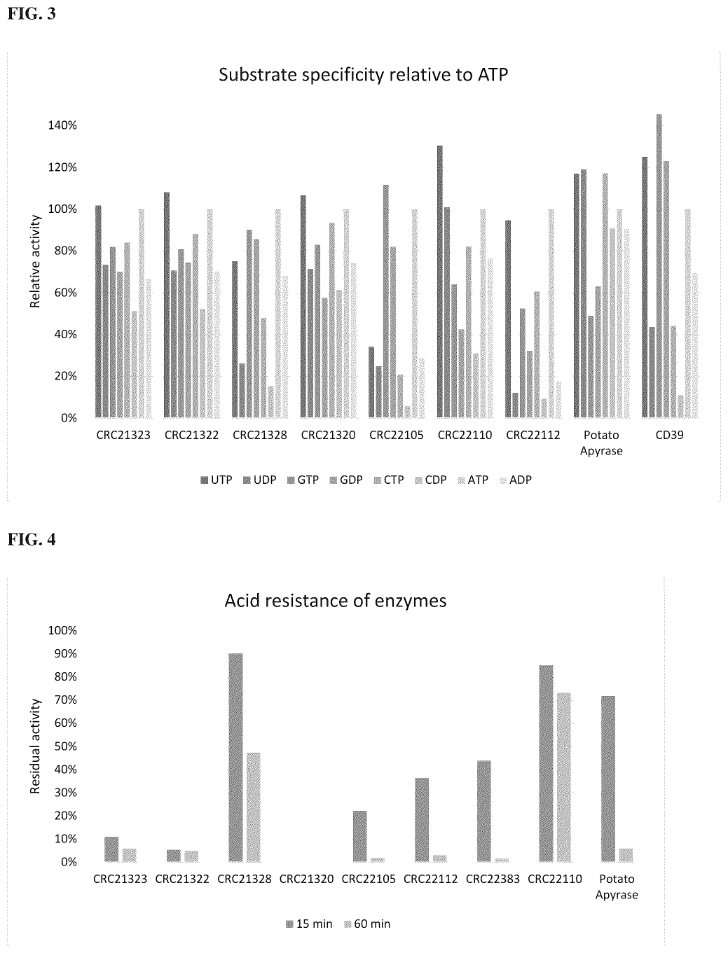

Enzyme activity towards ATP or ADP at pH 3 to 9: The ability of candidate enzymes to degrade ATP or ADP to AMP and free phosphate (Pi) was measured using a standard malachite green phosphate detection kit (Sigma Aldrich cat no. MAK307). The enzyme reactions were carried out in 96-well plates with a total volume of 150 μL and contained 250 μM of a substrate, 50 mM of a buffer, 5 mM CaCl₂, and a variable concentration of enzyme. The substrate was ATP or ADP. The buffer was glycine (pH 3), acetate (pH 4, 4.5, 5), bis-Tris (pH 5.5, 6, 6.5), or Tris (pH 7, 8, 9). The concentration of enzyme varied from about 1 μg/mL to 1 ng/mL and was adjusted based on the activity of the enzyme so that the amount of phosphate released during the assay was with the dynamic range of the assay (5-100 μM phosphate). The enzyme concentration was determined by the absorbance at 280 nm and using the predicted molecular weight and molar extinction coefficient to calculate the concentration. The enzyme reaction was initiated by the addition of 15 μL enzyme with 135 μL of the other reaction components such that the final reaction composition was as previously described. The reaction was stopped after 5 min by mixing 80 μL of the reaction mixture with 20 μL of the malachite green working reagent. The malachite green working reagent was prepared according to the manufacturer's protocol by mixing 100 parts reagent A with 1 part reagent B. Phosphate standard solutions (0-80 μM) and a negative control reaction without enzyme were also mixed with the malachite green working reagent. All samples were tested in duplicate. The malachite green reaction mixture was allowed to rest at room temperature for 45-60 min and the absorbance at 620 nm was measured. Lastly, the absorbance of the phosphate standards were used to convert the absorbance readings of each sample into phosphate concentrations and the negative control was subtracted from each reaction. The amount of phosphate in micromoles released per milligram of protein per minute of reaction time (μmol/mg/min) with ATP or ADP as a substrate is displayed in FIG. 1 and FIG. 2.

As shown in FIG. 1 and FIG. 2, CRC22110 was the most active enzyme. Potato apyrase, CRC22110, CRC22105, CRC22112, CRC22383, CRC21328 are members of the GDA1 CD39 superfamily. CRC21320 is a member of the purple acid phosphatase family. CRC21323 and CRC21322 are members of the acid phosphatase (class A) superfamily. The acid phosphatase enzymes have an acidic pH optimum around pH 4.5 whereas the enzymes from the GDA1_CD39 superfamily had pH optima near neutral pH (pH 6-8) and have a broad activity range between pH 5-7, with some maintaining activity even to pH 9. CRC21320 has a narrower pH range with an optimum at pH 7.

Enzyme substrate specificity: The activity of the enzymes on different nucleotide substrates was tested using the malachite green assay. The assay was set up as described in above with the following modifications. The substrates tested were ATP, ADP, CTP, CDP, UTP, UDP, GTP, and GDP. CRC21323 and CRC21322 were tested with a buffer of acetate pH 4.5. CD39 was tested with a buffer of bis-Tris pH 7. All other enzymes were tested with a buffer of bis-Tris pH 6.5. After converting the absorbance to phosphate concentration, the activity of each enzyme was set relative to the activity on ATP.

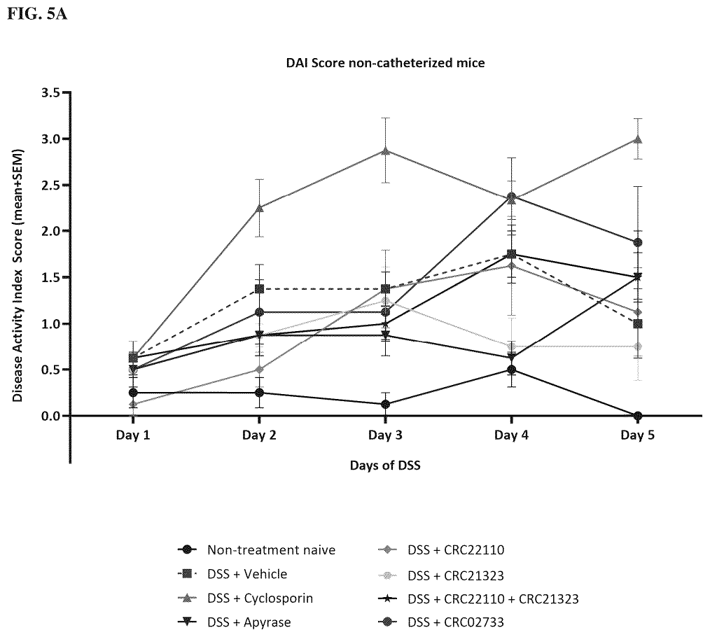

As shown in FIG. 3, the nucleotide triphosphate is a better substrate than the diphosphate. Most enzymes have similar activity on UTP as ATP, except for CRC22105 which has noticeably reduced activity on UTP/UDP and CTP/CDP. However, each enzyme has slightly difference preferences.

Enzyme acid resistance: Candidate enzymes were tested for their ability to resist inactivation at low pH by incubating the enzymes in an “acid buffer” of 50 mM glycine and 5 mM CaCl₂ at pH 2 for 15 or 60 minutes and comparing their activity to enzymes in a “control buffer” at their optimum pH. The optimum buffer for CRC21323 and CRC21322 was 50 mM acetate pH 4.5 with 5 mM CaCl₂. The optimum buffer for all other enzymes was 50 mM bis-Tris pH 6.5 with 5 mM CaCl₂. After incubating in the acidic glycine buffer, the enzyme mixtures were neutralized by mixing with an equal volume of neutralization buffer. The neutralization buffer for CRC21323 and CRC21322 was 100 mM acetate and 10 mM CaCl₂ at pH 4.5. The neutralization buffer for the other enzymes was 100 mM bis-Tris and 10 mM CaCl₂ at pH 6.5. Next, the acid-stressed and control enzyme were tested for their ability to release phosphate from ATP using the reaction conditions and malachite green assay as described in with the following changes. The only substrate tested was ATP. CRC21323 and CRC21322 were tested with a buffer of acetate pH 4.5, but all other enzymes were tested in a buffer of bis-Tris pH 6.5. After determining the concentration of phosphate in each reaction, the residual activity of each enzyme after 15 or 60 min incubation in the “acid buffer” was calculated by dividing the acid-stressed by the control enzyme phosphate concentrations for each enzyme.

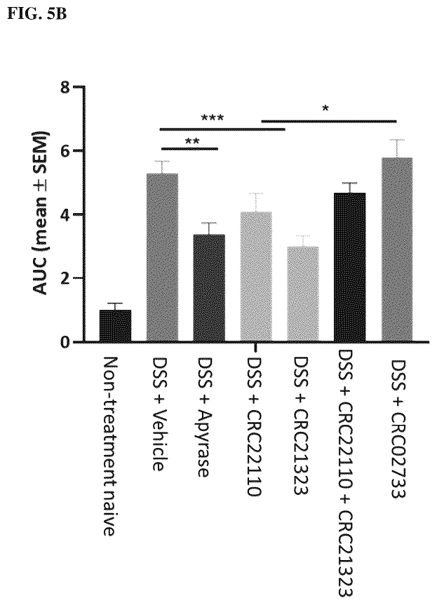

As shown in FIG. 4, the acid resistance experiment indicates that CRC21328 and CRC22110 had the greatest resistance to the “acid buffer” as they maintained the greatest activity after a 60 min incubation. Potato apyrase was stable after 15 min but appeared to lose most of its activity after 60 min. Although CRC21323 and CRC21322 are members of the acid phosphatase family, they were the least stable in this assay.

### Example 2: Use of Acid Phosphatases for Treatment of IBD in a Chemically-Induced DSS Colitis Mouse Model

Inflammatory bowel disease (IBD) is a complex multifactorial disease with contributing factors ranging from genetic mutations to micro and macro environment outside and inside the gut. Among a wide range of animal models of IBD, the chemically-induced dextran sulfate sodium (DSS) colitis model is well known for rapid screening of candidate compounds.

Acute DSS colitis was induced in C57BL/6 mice according to the previously published method with minor modification (Sann et al., 2013, Life Sci. April 9; 92(12):708-18). The mice were fed 3% (w/v) DSS dissolved in the drinking water on day one to day five. Fresh DSS solution was provided every other day. Control mice (Group 1) drank only distilled water. Groups 4 to 8 received candidate acid phosphatase enzymes by oral gavage and groups 9 to 12 received enzymes via catheter (Table 2). DAI was calculated as described earlier (Cooper et al., 1993, Laboratory investigation; a journal of technical methods and pathology. 69(2):238-49).

TABLE 2
Experimental design

| | | Dose | | Dose | | |
|---|---|---|---|---|---|---|
| | | Level | Dosing | volume | Concentration | No. of |
| Gr. | Group Treatment | (mg/kg) | frequency | (μL/mice) | (mg/mL) | animals |
| 1 | Non-treatment naive | — | — | — | — | 8 |
| 2 | DSS + Vehicle | 0 | PO; BID | 100 | N/A | 8 |
| 3 | DSS + Cyclosporin | 80 | QD | 10 mL/kg | 8 | 8 |
| | | (mg/mice) | | | | |
| 4 | DSS + Apyrase | 0.01 | PO; BID | 100 | 0.1 | 8 |
| 5 | DSS + CRC22110 | 0.0017 | PO; BID | 100 | 0.017 | 8 |
| 6 | DSS + CRC21323 | 0.01 | PO; BID | 100 | 0.1 | 8 |
| 7 | DSS + CRC22110 + | 0.0117 | PO; BID | 100 | 0.117 | 8 |
| | CRC21323 | | | | | |
| 8 | DSS + CRC02733 | 0.06 | PO; BID | 100 | 0.6 | 8 |
| * 9 | DSS + Catheter | 0 | BID | 100 | N/A | 8 |
| | control + Vehicle | | | | | |
| * 10 | DSS + Catheter + | 0.0017 | BID | 100 | 0.017 | 8 |
| | CRC22110 | | | | | |
| * 11 | DSS + Catheter + | 0.01 | BID | 100 | 0.1 | 8 |
| | CRC21323 | | | | | |
| * 12 | DSS + Catheter + | 0.0117 | BID | 100 | 0.117 | 8 |
| | CRC22110 + | | | | | |
| | CRC21323 | | | | | |

* Groups 9 to 12 received test items via cecum through catheterization.
BID: twice daily at 8 hrs interval.
QD: once daily
PO: oral gavage

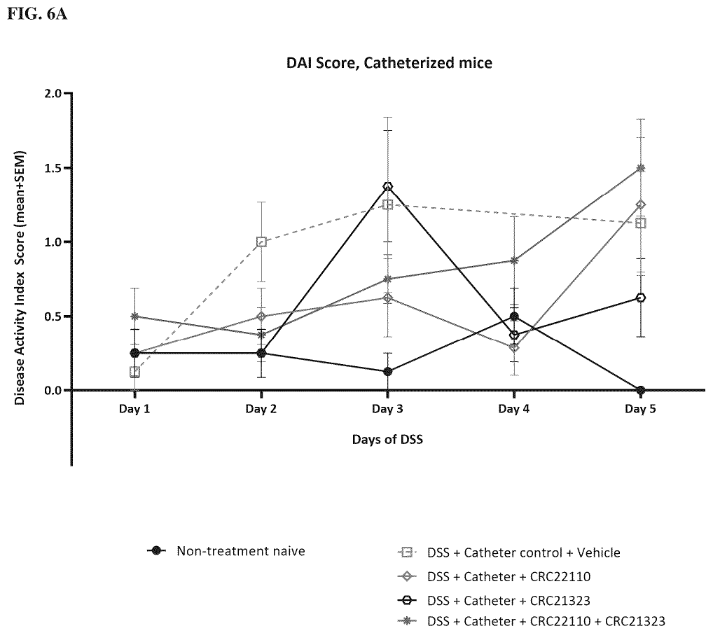

As shown in FIG. 5A and FIG. 5B, a significant improvement in DAI score was observed in mice receiving apyrase (improvement by 37%) and CRC21323 (improvement by 43%) compared to control. A significant reduction in DAI score was also observed for CRC22110 when compared to CRCO2733.

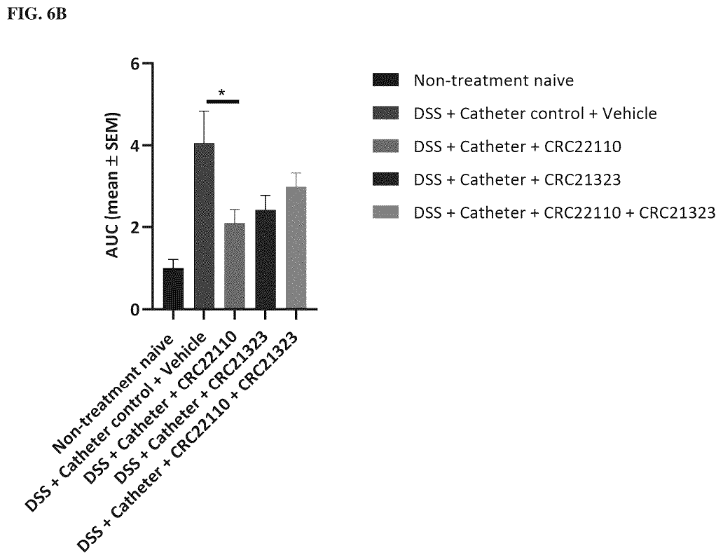

Furthermore, a significant improvement in DAI score (reduction by 47%) was observed in catheterized mice receiving CRC221100 compared to control. DAI scores were reduced by 40% and 25% in the groups receiving CRC21323 and the combination of CRC22110 and CRC21323 (FIG. 6A and FIG. 6B).

### Example 3: Use of Acid Phosphatases for Promoting Engraftment of Beneficial Gut Bacteria

Gut inflammation plays a major role in a number of disorders. It is a complex multifactorial disease with contributing factors ranging from genetic mutations to micro- and macro-environment both outside and inside the gut.

Apc/minJ mice have a mutated adenomatous polyposis coli gene and are more prone to develop colitis and tumors. In this Example, and using this model, 6-7 weeks old mice were subjected to an antibiotics cocktail of vancomycin, ampicillin, and neomycin (1 mg/mL) for 7 days in drinking water in order to induce a gut inflammatory response. Control mice (Groups 1 and 2) drank only distilled water. Groups 4 and 6 received CRC22110 ay 10 μg/dose enzyme by oral gavage and groups 5 and 6 received human microbiota transplant (HMT) by gavage. HMT was prepared by inoculating fecal sample from a healthy human donor in 20 ml of YCFAC media. Culture was grown anaerobically for 16 hrs at 37° C. Cells were collected by centrifugation and washed with PBS. Cells were resuspended in 10% glycerol and cell numbers were estimated by plating on YCFAC agar plates.

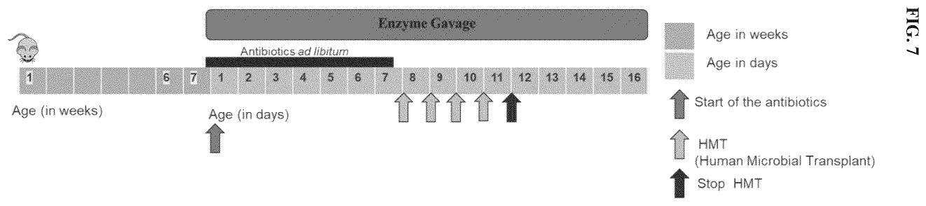

Experiments design is shown in Table 3 and FIG. 7.

| | | | | | | | Human Microbiota | | | |
|---|---|---|---|---|---|---|---|---|---|---|
| | | | Antibiotic | Enzyme (Enz)^{a} | | | Transfer (HMT)^{b} | | | Number |
| | | Group | mix (Ab) | Dose | Volume | | Dose | Volume | | of |
| Group | Genotype | Treatment | Route | (mg/kg) | (uL) | Route | (mg/kg) | (uL) | Route | animals |
| 1^{c} | Wild type | No Ab, | — | — | — | PO | — | — | PO | 6 |
| | C57BL/6 | No Enz, | Sterile | vehicle | 50 | | vehicle | 100 | | |
| | | No HMT | drinking | | | | | | | |
| | | | water | | | | | | | |
| 2^{c} | C57BL/6J | No Ab, | — | — | — | PO | — | — | PO | 6 |
| | ApcMin/J | No Enz, | Sterile | vehicle | 50 | | vehicle | 100 | | |
| | | No HMT | drinking | | | | | | | |
| | | | water | | | | | | | |
| 3^{c} | | Ab | Sterile | — | — | PO | — | — | PO | 6 |
| | | | drinking | vehicle | 50 | | vehicle | 100 | | |
| | | | water ad | | | | | | | |
| | | | libitum | | | | | | | |
| 4 | | Ab + | Sterile | 10 ug/ | 50 | PO | — | — | PO | 6 |
| | | Enz | drinking | dose | | | vehicle | 100 | | |
| | | | water ad | | | | | | | |
| | | | libitum | | | | | | | |
| 5 | | Ab + | Sterile | — | — | PO | 10⁸ | 100 | PO | 6 |
| | | HMT | drinking | vehicle | 50 | | cfu/dose | | | |
| | | | water ad | | | | | | | |
| | | | libitum | | | | | | | |
| 6 | | Ab + | Sterile | 10 ug/ | 50 | PO | 10⁸ | 100 | PO | 6 |
| | | HMT + | drinking | dose | | | cfu/dose | | | |
| | | Enz | water ad | | | | | | | |
| | | | libitum | | | | | | | |

^{a}the enzyme was given orally (PO) twice per day from day 1 to day 16
^{b}HMT was given PO once per day from day 9 to day 12. For group 6, Enzyme will be given first.
^{c}Groups 1, 2 and 3 was given PO twice per day from day 1 to day 16. Groups 1, 2 and 3 will receive an extra PO from day 9 to 12 to match HMT dosage.

Disease Severity: Disease severity was scored using a clinical disease activity index (DAI), calculated as previously described using the following parameters: stool consistency, presence or absence of fecal blood and weight loss (Cooper et al., 1993, Laboratory investigation; a journal of technical methods and pathology. 69(2):238-49). The mice were killed on days 12 and 16, colons were measured, and the tissue was prepared for histopathological studies. The colon was fixed in 10% formaldehyde-saline. Hematoxylin and eosin stain (HE)-stained sections are graded based on a scoring system adapted from Burich et al., 2001, Am. J. Physiol. Gastrointest. Liver Physiol. 281:G764-G778 and Hausmann et al., 2007, Clin Exp Immunol. 2007 May; 148(2):373-81. Histology scoring is performed, and a combined score of inflammatory cell infiltration and tissue damage is determined for all the groups.

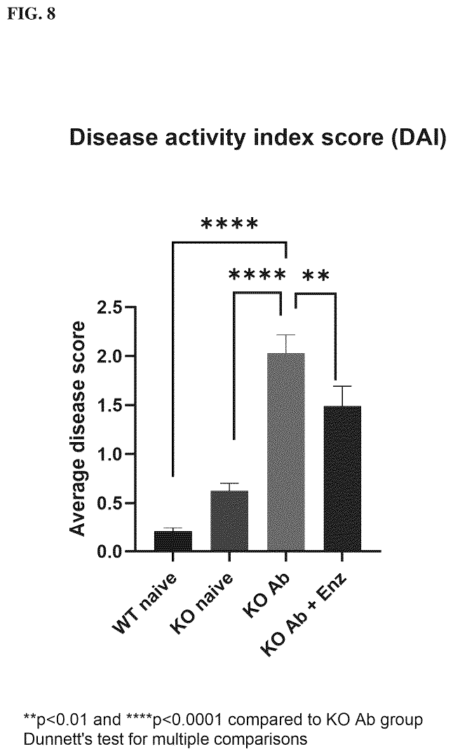

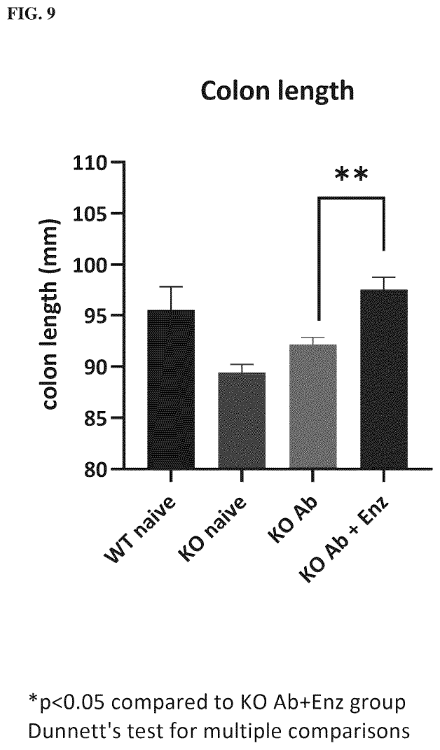

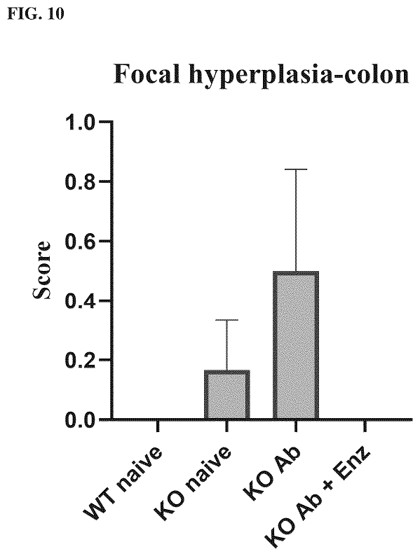

As shown in FIG. 8, a significant improvement in DAI score was observed in mice receiving CRC22110 (improvement by 25%) as compared to control. Stool consistency and fecal hemoccult score changes were apparent 2 days after treatment, with no decrease in body weight. Disease activity score (DAI) reached a maximum of 2-3. As shown in FIG. 9, a significant improvement in colon length was observed in mice receiving CRC22110 as compared to control. Further, as shown in FIG. 10, histopathologic analysis showed that the focal hyperplasia observed in control groups was absent in group receiving CRC22110. A hyperplastic polyp is a growth of extra cells that projects out from tissues. They occur in areas where body has repaired damaged tissue, especially along digestive tract. Absence of hyperplasia in the group receiving CRC22110 indicated that the enzyme was able to prevent the damage on the GI tract under inflamed conditions.

Promotion of Engraftment: Fecal samples were collected from each mouse per day. Day 13 fecal samples were processed and 16S sequencing was done to evaluate the structure of microbiome. Group 5 was compared with group 6 and engraftment of HMT was evaluated. HMT was prepared by inoculating fecal sample from a healthy human donor in 20 ml of YCFAC media. Culture was grown anaerobically for 16hrs. Cells were collected by centrifugation and washed with PBS. Cells were resuspended in 10% glycerol and cell numbers were estimated by plating.

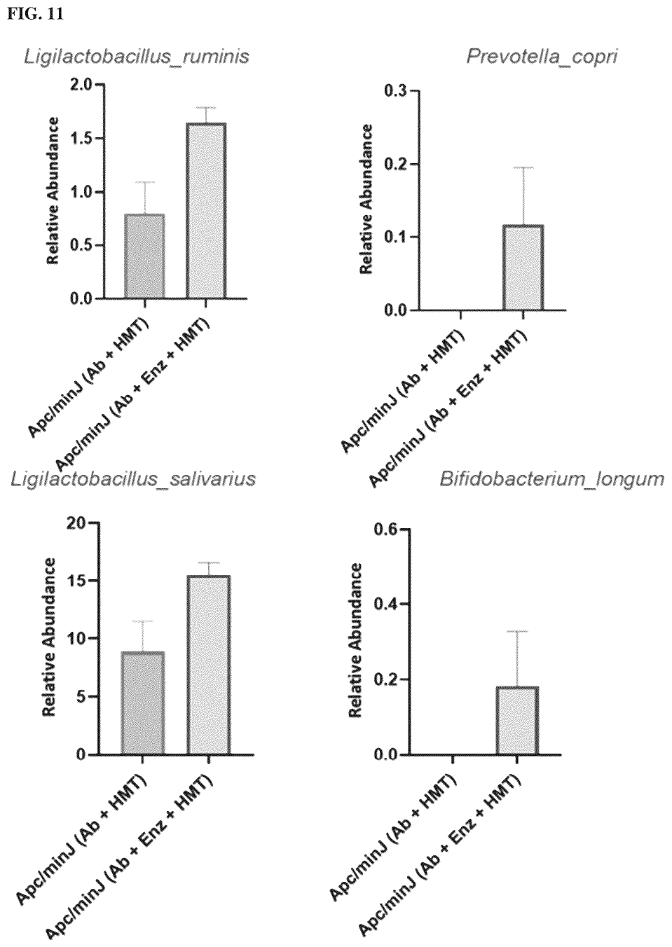

As shown in FIG. 11, animals that received CRC22110 showed better engraftment of several commensals from HMT in group 6.

### Example 4. Cloning, Expression and Protein Preparation of Enzyme Candidates

Phylogenetically diverse sequences from enzyme families known to have ATP hydrolysis activity were identified from the NCBI (worldwwideweb.ncbi.nlm.nih.gov/protein) or internal databases and annotated as members of the enzyme families GDA1/CD39 or acid phosphatase (class A). Gene sequences of interest were codon-optimized based on codon preference of Bacillus subtilis and synthetic genes encoding the sequences for various enzymes were generated (by Generay Biotech (Shanghai) Co., Ltd) using techniques known in the art. The sequences of codon optimized genes and predicted mature enzymes are listed on Table 4. Genes were cloned into expression vector p2JM103BBI (Vogtentanz, Protein Expr Purif. 55:40-52, 2007). The expression of the codon-optimized nucleotide sequence encodes the predicted mature polypeptide driven by aprE promoter (SEQ ID NO: 3), using the aprE signal peptide (SEQ ID NO: 4) with an additional 3 amino acids (Ala-Gly-Lys) between the signal sequence and the predicted mature sequence of the genes of interest (to facilitate the secretion of the target protein in B. subtilis), and is stopped by lat terminator (SEQ ID NO: 5). Competent B. subtilis cells were transformed with each corresponding expression vector and plated on Luria Agar plates supplemented with 5 ppm chloramphenicol. The colony was inoculated in a flask containing LB medium and grew at 37° C. with 200 rpm shaking for 5-6 h. Seed culture was inoculated into a production medium containing minerals (e.g., potassium sulfate, magnesium sulfate, ferrous sulfate, calcium chloride, citric acid, etc.), one or more carbon sources (e.g., glucose), a complex nitrogen source (e.g. hydrolyzed soy peptone, yeast extract) and a buffering component (e.g. MOPS, Tris, etc.). Cultures were grown at 32° C. with 250 rpm shaking for 24-40 h.

General purification procedure was as follows. Culture broth was collected by centrifugation, concentrated and supplemented with ammonium sulfate to a final concentration of 1M, then applied to a hydrophobic interaction column (e.g. phenyl FF, butyl FF) which was pre-equilibrated with 50 mM Tris pH 7.5 containing 1M ammonium sulfate. The column was eluted with a stepwise mode (0.75, 0.5, 0.25, 0.02 M ammonium sulfate, H₂O, 20% ethanol) and fractions containing active enzymes of interest were pooled and buffer-exchanged to 20 mM Tris pH 7.5. Pooled sample was loaded on an anion-exchange chromatography (e.g. Q FF) and eluted with a gradient mode (0-0.5 M NaCl). Fractions containing purified enzymes were identified based on SDS-PAGE analysis and activity tests, and they were pooled. Purified samples were stored at −20° C. in buffer containing 20 mM Tris pH 7.5, 150 mM NaCl, and 40% glycerol until use.

Reference enzymes were purchased from Sigma Aldrich: Potato apyrase (catalog #A6535), recombinant human CD39 His-tag (catalog #SRP0623), and calf intestinal alkaline phosphatase (catalog #P0114).

TABLE 4
Family designation, accession numbers and sequence listing numbers for enzymes
of interest. SEQ ID (protein) refers to predicted mature protein sequences,
and SEQ ID (DNA) refers to nucleotide sequences of codon optimized genes.

| Enzyme | | | SEQ ID | SEQ ID |
|---|---|---|---|---|
| identifier | Assigned enzyme family | Accession number | (protein) | (DNA) |
| CRC21328 | GDA1/CD39 | CCD05982.1 | 6 | 25 |
| CRC22104 | GDA1/CD39 | TAL58835.1 | 7 | 26 |
| CRC22105 | GDA1/CD39 | WP_045094910.1 | 8 | 27 |
| CRC22110 | GDA1/CD39 | EKE69473.1 | 9 | 28 |
| CRC22112 | GDA1/CD39 | KTD66016.1 | 10 | 29 |
| CRC22383 | GDA1/CD39 | WP_058475114.1 | 11 | 30 |
| CRC22386 | GDA1/CD39 | WP_173236575.1 | 12 | 31 |
| CRC22387 | GDA1/CD39 | WP_127099836.1 | 13 | 32 |
| CRC22605 | GDA1/CD39 | WP_133471262.1 | 14 | 33 |
| CRC22606 | GDA1/CD39 | OQW93475.1 | 15 | 34 |
| CRC22620 | GDA1/CD39 | NBS62745.1 | 16 | 35 |
| CRC21322 | Acid phosphatase (Class A) | AEG96708.1 | 17 | 36 |
| CRC21323 | Acid phosphatase (Class A) | WP_054306375.1 | 18 | 37 |
| CRC25082 | Acid phosphatase (Class A) | WP_050460978.1 | 19 | 38 |
| CRC25084 | Acid phosphatase (Class A) | WP_088823167.1 | 20 | 39 |
| CRC25085 | Acid phosphatase (Class A) | WP_092747651.1 | 21 | 40 |
| CRC25089 | Acid phosphatase (Class A) | WP_217352265.1 | 22 | 41 |
| CRC25099 | Acid phosphatase (Class A) | WP_195761705.1 | 23 | 42 |
| CRC25100 | Acid phosphatase (Class A) | internal database | 24 | 43 |

### Example 5. Characterization of Additional Enzyme Candidates

Enzymes of interest were assayed against ATP substrate. The ATPase activity was measured at 0.05 ppm concentration in 50 mM IVIES buffer pH 6 and pH 5 with 0.1 mM ATP as substrate as described in Example 1. Commercial potato apyrase was used as reference. The absorbance at 620 nm was measured and the absorbance of a blank sample (water) was subtracted from each sample. Results are shown on Table 5.

TABLE 5
Activity of enzymes tested at 0.05 ppm on 0.1 mM ATP substrate.

| Sample ID | Enzyme family | pH 6.0 | pH 5.0 |
|---|---|---|---|
| CRC21328 | GDA1/CD39 | 0.27 | 0.45 |
| CRC22104 | GDA1/CD39 | 0.10 | 0.05 |
| CRC22105 | GDA1/CD39 | 0.28 | 0.26 |
| CRC22110 | GDA1/CD39 | 0.37 | 0.60 |
| CRC22112 | GDA1/CD39 | 0.06 | 0.04 |
| CRC22383 | GDA1/CD39 | 0.10 | 0.07 |
| CRC22386 | GDA1/CD39 | 0.25 | 0.36 |
| CRC22387 | GDA1/CD39 | 0.11 | 0.36 |
| CRC22605 | GDA1/CD39 | 0.28 | 0.50 |
| CRC22606 | GDA1/CD39 | 0.21 | 0.43 |
| CRC22612 | GDA1/CD39 | 0.23 | 0.09 |
| CRC22620 | GDA1/CD39 | 0.22 | 0.13 |
| CRC21322 | Acid phosphatase (Class A) | 0.07 | 0.40 |
| CRC21323 | Acid phosphatase (Class A) | 0.10 | 0.39 |
| CRC25082 | Acid phosphatase (Class A) | 0.65 | 0.93 |
| CRC25084 | Acid phosphatase (Class A) | 0.17 | 0.20 |
| CRC25085 | Acid phosphatase (Class A) | 0.20 | 0.23 |
| CRC25089 | Acid phosphatase (Class A) | 0.05 | 0.24 |
| CRC25099 | Acid phosphatase (Class A) | 0.93 | 1.05 |
| CRC25100 | Acid phosphatase (Class A) | 0.90 | 1.11 |
| Potato Apyrase | GDA1/CD39 | 0.43 | 0.45 |

The pH profile of additional enzymes of interest was assayed using 1 mM ATP, in 50 mM trisodium citrate/citric acid buffer with 5 mM CaCl2. Absorbance was read at 620 nm and all values were set relative to the highest one. Commercial potato apyrase was used as reference. The relative activity of these enzymes of interests tested between pH 3.5 and pH 6.5 is shown on Table 6.

TABLE 6
Relative activity of enzymes on ATP between pH 3.5 to 6.5

| Sample ID | pH 3 | pH 3.5 | pH 4 | pH 4.5 | pH 5 | pH 5.5 | pH 6 | pH 6.5 |
|---|---|---|---|---|---|---|---|---|
| CRC22104 | 0.0 | 0.0 | 0.0 | 0.3 | 0.8 | 0.9 | 1.0 | 1.0 |
| CRC22386 | 0.0 | 0.0 | 0.0 | 0.0 | 0.2 | 0.6 | 0.8 | 1.0 |
| CRC22387 | 0.1 | 0.0 | 0.0 | 0.1 | 0.3 | 0.5 | 1.0 | 1.0 |
| CRC2252 | 0.0 | 0.0 | 0.0 | 0.2 | 0.7 | 0.9 | 0.9 | 1.0 |
| CRC22605 | 0.0 | 0.0 | 0.0 | 0.0 | 0.4 | 0.8 | 1.0 | 1.0 |
| CRC22606 | 0.0 | 0.0 | 0.0 | 0.3 | 0.6 | 0.8 | 0.9 | 1.0 |
| CRC22612 | 0.0 | 0.0 | 0.0 | 0.0 | 0.1 | 0.3 | 0.6 | 1.0 |
| CRC22620 | 0.0 | 0.0 | 0.0 | 0.1 | 0.4 | 0.7 | 1.0 | 0.8 |
| CRC25082 | 0.0 | 0.0 | 0.0 | 0.1 | 0.3 | 0.7 | 1.0 | 0.8 |
| CRC25084 | 0.4 | 0.6 | 0.6 | 0.9 | 0.9 | 1.0 | 1.0 | 0.9 |
| CRC25085 | 0.5 | 0.6 | 0.8 | 0.9 | 0.9 | 1.0 | 0.8 | 0.9 |
| CRC25089 | 0.8 | 1.0 | 0.9 | 0.9 | 0.8 | 0.7 | 0.3 | 0.2 |
| CRC25099 | 0.0 | 0.1 | 0.3 | 0.7 | 0.8 | 1.0 | 0.8 | 0.5 |
| CRC25100 | 0.0 | 0.0 | 0.1 | 0.4 | 0.8 | 1.0 | 1.0 | 0.7 |
| potato | 0.0 | 0.0 | 0.1 | 0.4 | 0.6 | 0.8 | 1.0 | 0.9 |
| Apyrase | | | | | | | | |

### Example 6. Sequence Analysis of GDA1_CD39 Enzymes of Interest

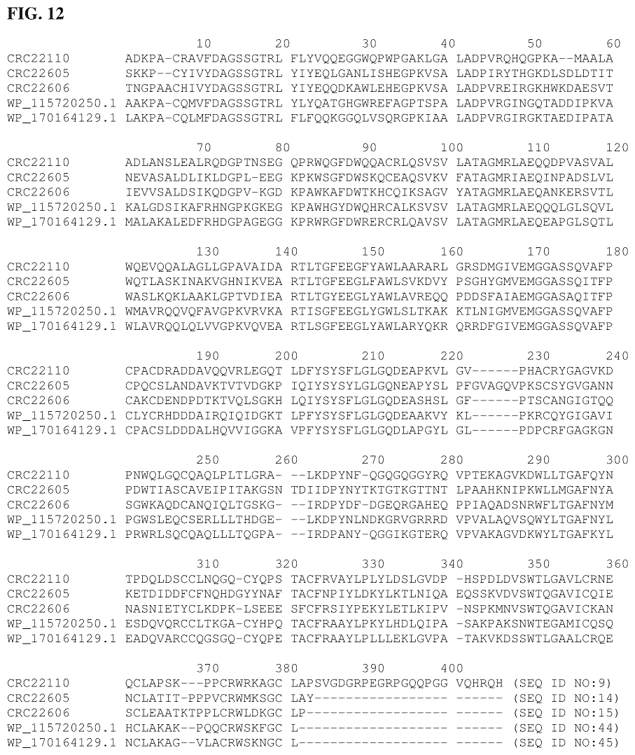

A multiple protein sequence alignment was performed using Clustal W with default settings (in Geneious Prime software tool) for the GDA1/CD39 family members: CRC22110 (SEQ ID NO:9), CRC22605 (SEQ ID NO: 36), CRC22606 (SEQ ID NO: 37), WP_115720250.1 (SEQ ID NO: 48) and WP_170164129.1 (SEQ ID NO: 49). The alignment of residues spanning the sequence of the predicted mature CRC22110 protein is shown on FIG. 12 which highlights the sequence similarities among these homologous enzymes. The percent identity of this group of enzymes is shown on Table 7, which includes the apyrase from Legionella pneumophila (PDB ID: 4BRA, SEQ ID NO: 50) for comparison.

TABLE 7
Protein sequence comparison for CD39 enzymes.

| Identifier | % Identity to CRC22110 |
|---|---|
| CRC22605 | 43.6 |
| CRC22606 | 44.3 |
| WP_115720250.1 | 51.8 |
| WP_170164129.1 | 59.1 |
| pdb\|4BRA\|A | 21.1 |

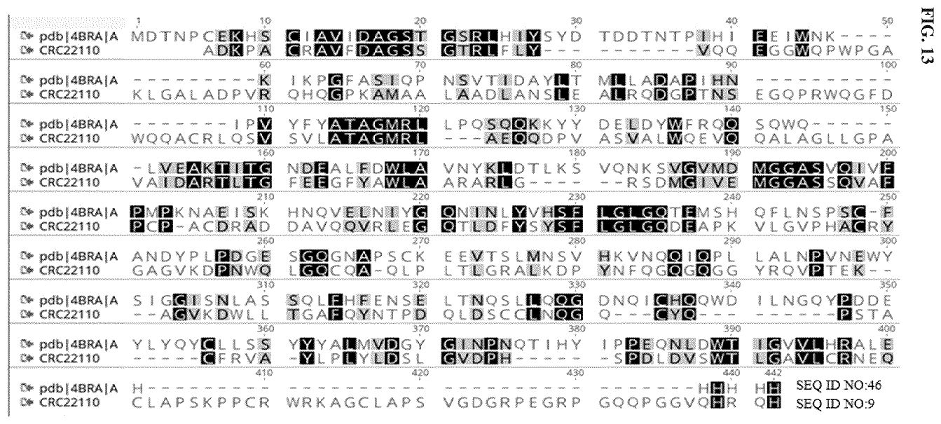

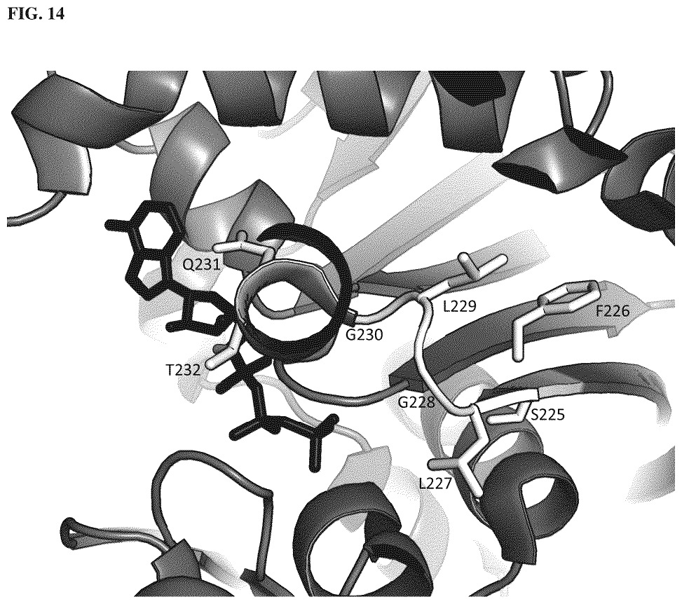

A pairwise protein sequence alignment of CRC22110 (SEQ ID NO:9) and the apyrase from Legionella pneumophila (PDB ID: 4BRA, SEQ ID NO: 50) was performed using Clustal W to identify the conserved regions shared by these CD39 enzymes. The alignment is shown on FIG. 13, where the positions are numbered according to the sequence of 4BRA (accounting for the sequence gaps introduced by the alignment) and it shows tracks of sequence homology even though the protein sequences are significantly different (21% identity). The image on FIG. 14 was generated using Pymol software (version 2.4.1) for the Legionella pneumophila apyrase PDB ID: 4BRA structure and highlights a region that is critical for ATP hydrolysis activity that is adjacent to the active site which is occupied by an ANP molecule (black sticks). A series of single-site variants of CRC22110 were generated in order to evaluate the functional significance of the corresponding residues within this region in the CRC22110 molecule. The variants were tested for activity on ATP substrate in comparison to CRC22110 wild type, and the relative activity results are shown on Table 8. Substitutions at positions corresponding to 225, 226, 227, 228, 229 and 230 of the Legionella pneumophila apyrase (4BRA) result in loss of enzyme activity in all CRC22110 variants, consistent with the hypothesis that this conserved region is important for activity even when the apyrase enzymes differ greatly in overall sequence identity.

TABLE 8
Relative activity on ATP for CRC22110 and single
site variants in region adjacent to active site.

| | Corresponding position | Mutation site in | Relative |
|---|---|---|---|
| | in 4BRA sequence | CRC22110 | activity |
| Enzyme name | (SEQ ID NO: 50) | (SEQ ID NO: 9) | (%) |
| CRC22110v1 | S225 | S204A | 37 |
| CRC22110v7 | S225 | S204T | 53 |
| CRC22110v8 | F226 | F205A | 68 |
| CRC22110v9 | F226 | F205L | 68 |
| CRC22110v2 | L227 | L206A | 60 |
| CRC22110v10 | L227 | L206I | 16 |
| CRC22110v3 | G228 | G207A | 60 |
| CRC22110v4 | L229 | L208A | 74 |
| CRC22110v5 | G230 | G209A | 16 |
| CRC22110-WT | | Wild type | 100 |

### Example 7. Sequence Analysis of Acid Phosphate (Class A) Enzymes of Interest

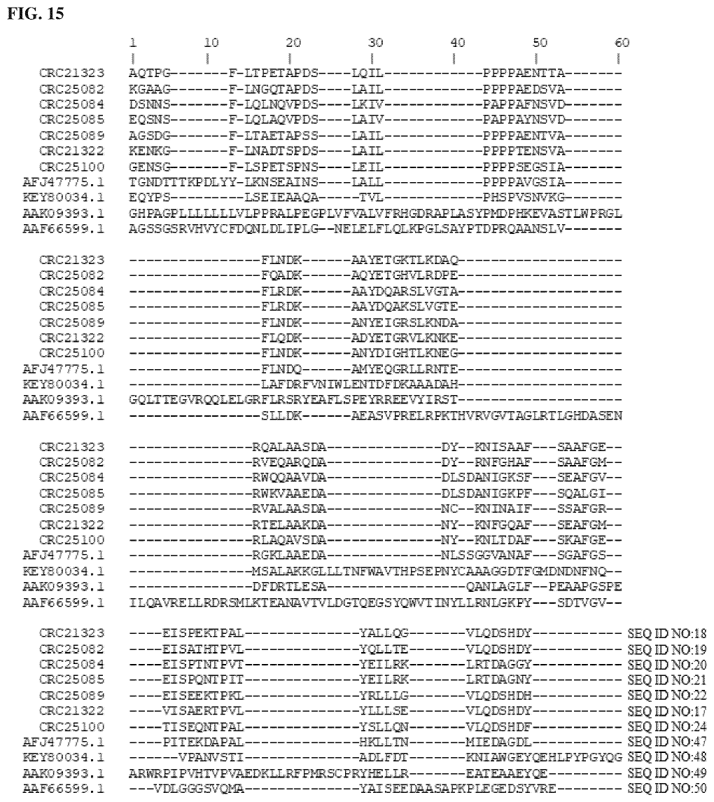

In order to evaluate the structure-function relationship among bacterial acid phosphatases, a multiple protein sequence alignment was performed using Clustal W software with default settings (in Geneious Prime). Bacterial acid phosphates evaluated in this study: CRC21323 (SEQ ID NO:41), CRC25082 (SEQ ID NO:42), CRC25084 (SEQ ID NO:43), CRC25085 (SEQ ID NO:44), CRC25089 (SEQ ID NO:45), CRC21322 (SEQ ID NO:40), and CRC25100 (SEQ ID NO:47) we aligned with another bacterial acid phosphatase from Escherichia blattae (PDB ID: 1D2T, sequence AFJ47775.1 (SEQ ID NO:51)), and three more divergent enzymes from eukaryotes: Aspergillus fumigatus KEY80034.1 (SEQ ID NO:52), Homo sapiens AAK09393.1(SEQ ID NO:53), and Arabidopsis thaliana AAF66599.1 (SEQ ID NO:54) were included for comparison. The alignment of residues spanning the sequence of the predicted mature CRC21323 protein is shown on FIG. 15 which highlights the sequence similarities among the homologous bacterial enzymes. The percent identity of this group of enzymes is shown on Table 9.

TABLE 9
Protein sequence comparison for acid phosphate enzymes.

| | Percent sequence |
|---|---|
| Identifier | identity to CRC21323 |
| CRC25082 | 70.1 |
| CRC25084 | 50.5 |
| CRC25085 | 50.5 |
| CRC25089 | 75.2 |
| CRC21322 | 72.9 |
| CRC25100 | 74.3 |
| AFJ47775.1 | 52.5 |
| KEY80034.1 | 11.1 |
| AAK09393.1 | 10.4 |
| AAF66599.1 | 12.2 |

The bacterial acid phosphatases show a 50% or greater sequence identity.

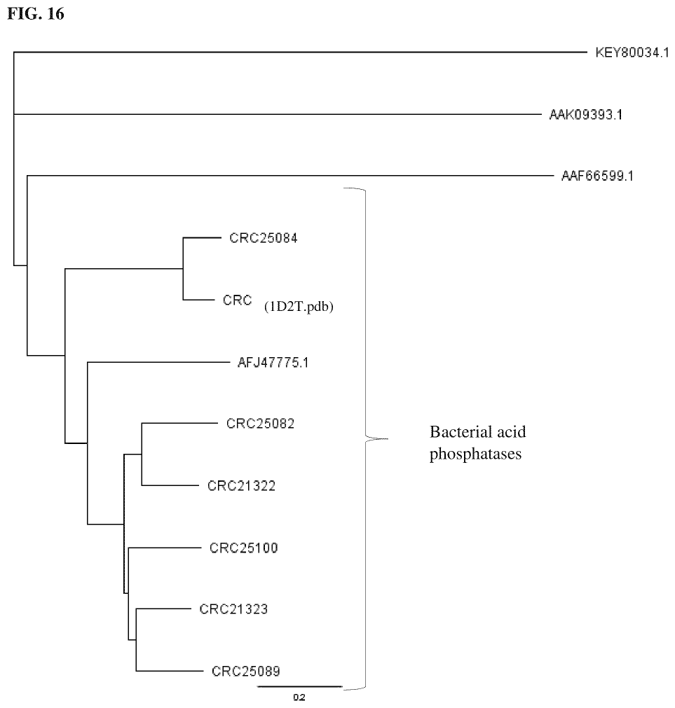

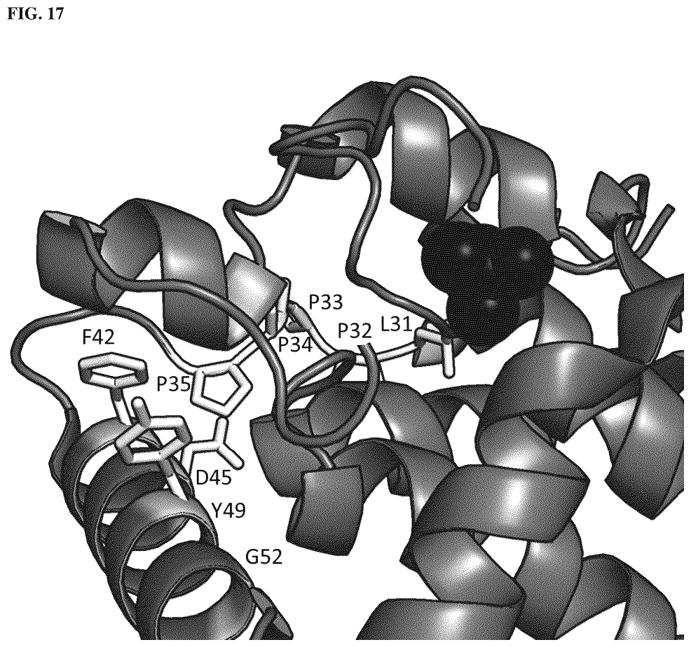

A phylogenetic tree for the sequences in FIG. 15 was constructed using the neighbor-joining method (MEGA X program) and is shown on FIG. 16, where it can be seen that the bacterial enzymes are clustered in one Glade. A region of high sequence identity among the bacterial acid phosphatases can also be seen in the 3D structure of the apyrase from Escherichia blattae (PDB ID: 1D2T). As shown on FIG. 17, in an image generated using Pymol (version 2.4.1) the region near the active site pocket (indicated by the phosphate molecule) forms a flexible loop and an alpha-helix, with its location indicating critical function for ATP hydrolysis. Amino acid sequences within this region and flanking it appear highly conserved among the bacterial acid phosphatases CRC21323, CRC25082, CRC25084, CRC25085, CRC25089, CRC21322, CRC25100, and AFJ47775.1.

SEQUENCES

MGWIRGLVALLLGLAWAVQAADKPACRAVEDAGSSGTRLFLYVQQEGGW
QPWPGAKLGALADPVRQHQGPKAMAALAADLANSLEALRQDGPTNSEGQ
PRWQGFDWQQACRLQSVSVLATAGMRLAEQQDPVASVALWQEVQQALAG
LLGPAVAIDARTLTGFEEGFYAWLAARARLGRSDMGIVEMGGASSQVAF
PCPACDRADDAVQQVRLEGQTLDFYSYSFLGLGQDEAPKVLGVPHACRY
GAGVKDPNWQLGQCQAQLPLTLGRALKDPYNFQGQGQGGYRQVPTEKAG
VKDWLLTGAFQYNTPDQLDSCCLNQGQCYQPSTACFRVAYLPLYLDSLG
VDPHSPDLDVSWTLGAVLCRNEQCLAPSKPPCRWRKAGCLAPSVGDGRP
EGRPGQQPGGVQHRQH (SEQ ID NO: 1)
MRLHFKKRVAVCSVAALLFSASGAFAAQTPGFLTPETAPDSLQILPPPP
AENTTAFLNDKAAYETGKTLKDAQRQALAASDADYKNISAAFSAAFGEE
ISPEKTPALYALLQGVLQDSHDYAMRAAKDHYMRVRPFVVYKDSTCTPE
KDKSMAKTGSYPSGHASFGWATALVLTEINPARETEILKRGYDFGQSRV
VCGAHWQSDVDNGRLMGAAVVAALHGNQGFNDALAKAKAEFAKR
(SEQ ID NO: 2)

1. A composition comprising a) an enzyme that hydrolyzes nucleotide triphosphates, wherein said enzyme is active at least from about pH 3.5 to pH 7; and b) an enteric coating.

2. The composition of claim 1, wherein the enzyme 1) is a member of the GDA1_CD39 superfamily; and 2) a) is not potato apyrase; and/or b) is not a mammalian NTPDase.

3. The composition of claim 1, wherein the enzyme comprises a polypeptide at least 40% identical to the amino acid sequence of SEQ ID NO:1 or SEQ ID NO:9 (CRC22110).

4. The composition of claim 1, wherein the enzyme is 1) an acid phosphatase (EC 3.1.3.2) and; 2) is not derived from Shigella.

5. The composition of claim 4 wherein the enzyme comprises a polypeptide at least 50% identical to the amino acid sequence of SEQ ID NO:2 or SEQ ID NO:18 (CRC21323).

6. The composition of claim 1, wherein the nucleotide triphosphate comprises ATP.

7. (canceled)

8. (canceled)

9. The composition of claim 1, wherein the enzyme hydrolyzes ATP with a specific activity of at least about 500 μmol/mg/min to about 2700 μmol/mg/min from about pH 3.5 to about pH 7.

10. The composition of claim 1, wherein the enteric coating is for 1) oral administration; 2) delivery to the intestinal mucosa; and 3) protection from degradation of the enzyme at a pH of less than about 2.5.

11. The composition of claim 1, wherein the enteric coating is formulated for rectal administration.

12. A nucleic acid encoding the enzyme of claim 1.

13. A vector comprising the nucleic acid of claim 12.

14. A recombinant host cell comprising the enzyme of claim 1.

15. The cell of claim 14, wherein the cell is a plant cell, a bacterial cell, a fungal cell, or a yeast cell.

16. The cell of claim 15, wherein the cell is a Bacillus subtilis cell, an E. coli cell, a Yarrowia cell, an Aspergillus niger cell, or a Trichoderma reesei cell.

17. A method for decreasing or preventing a disorder characterized by inflammation in the gut in a subject in need thereof, comprising administering to said subject the composition of claim 1.

18. The method of claim 17, wherein the disorder is a gastro-intestinal (GI) tract inflammatory disease selected from the group consisting of inflammatory bowel disease (IBD), Crohn's disease, colitis, colitis ulcerosa, enterocolitis, and necrotising enterocolitis.

19. (canceled)

20. A method for promoting the engraftment of one or more commensal bacteria into the gut of a subject, said method comprising administering to said subject the composition of claim 1 and a source of one or more commensal bacteria.

21. The method of claim 20, wherein said administration is oral administration.

22. The method of claim 20, wherein said administration of the enzyme is oral administration and administration of the source of one or more commensal bacteria is rectal administration.

23. The method of claim 21, wherein said subject has been diagnosed with a disorder characterized by inflammation in the gut.

24. The method of claim 23, wherein the disorder is a gastro-intestinal (GI) tract inflammatory disease selected from the group consisting of inflammatory bowel disease (IBD), Crohn's disease, colitis, colitis ulcerosa, enterocolitis, and necrotising enterocolitis.

25. (canceled)

26. The method of claim 20, wherein the source of one or more commensal bacteria comprises a probiotic.

27. The method of claim 20, wherein the source of one or more commensal bacteria comprises a fecal microbiota transplant (FMT).

28. The method of claim 20, wherein the one or more commensal bacteria is selected from the group consisting of Ligilactobacillus ruminis, Prevotella copri, Ligilactobacillus salivarius, and Bifidobacterium longum.


SEQUENCE LISTING (50 sequences)

SEQ ID NO: 1 - protein, 408 aa, Gallaecimonas xiamenensis
MGWIRGLVALLLGLAWAVQAADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSVGDGRPEGRPGQQPGGVQHRQH

SEQ ID NO: 2 - protein, 240 aa, Serratia rubidaea
MRLHFKKRVAVCSVAALLFSASGAFAAQTPGFLTPETAPDSLQILPPPPAENTTAFLNDKAAYETGKTLKDAQRQALAASDADYKNISAAFSAAFGEEISPEKTPALYALLQGVLQDSHDYAMRAAKDHYMRVRPFVVYKDSTCTPEKDKSMAKTGSYPSGHASFGWATALVLTEINPARETEILKRGYDFGQSRVVCGAHWQSDVDNGRLMGAAVVAALHGNQGFNDALAKAKAEFAKR

SEQ ID NO: 3 - DNA, 607 bp, Bacillus subtilis
aattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 4 - DNA, 96 bp, Bacillus subtilis
gtgagaagcaaaaaattgtggatcagcttgttgtttgcgttaacgttaatctttacgatggcgttcagcaacatgagcgcgcaggcagctggtaaa

SEQ ID NO: 5 - DNA, 30 bp, Bacillus licheniformis
cggatttcctgaaggaaatccgttttttta

SEQ ID NO: 6 - protein, 359 aa, Legionella pneumophila
DTNPCEKHSCIAVIDAGSTGSRLHIYSYDTDNTNTPIHIEEIWNKKIKPGFASIQPNSVTIDAYLTMLLADAPIHNIPVYFYATAGMRLLPQSQQKKYYDELEYWFRQQSQWQLVEAKTITGNDEALFDWLAVNYKLDTLKSVQNKSVGVMDMGGASVQIVFPMPKNAEISKHNQVELNIYGQNINLYVHSFLGLGQTEMSHQFLNSPSCFANDYPLPDGESGQGNAPSCKEEVTSLMNSVHKVNQQIQPLLALNPVNEWYSIGGISNLASSQLFHFENSELTNQSLLQQGDNQICHQQWDILNGQYPDDEYLYQYCLLSSYYYALMVDGYGINPNQTIHYIPPEQNLDWTIGVVLHRA

SEQ ID NO: 7 - protein, 366 aa, Legionella sp.
IEQDPCQEKQCMIVVDAGSTGSRLHFYTYDTDETNSPINIEEIWSKRIKPGISHVEANRSSIDAYLVTLLAGTPHSNHVPVYFYATAGMRLLPAAKQKLIYQEIKFWFAQQNQWRLMDAKTIYGSQEALYDWLAVNHQLGTLQSVQTKSVGVMDMGGASVQIVFPVQNNPDLKNNTLAEFNLYGQHIRLAVRSFLGLGQTEMSHQFLNYAPCFANNYPLPDGFLGEGDALSCVEEVASLMNGVHSVNKEIQPLLAANPVNIWYAIGGLSFLADNSVIHFNNYQLTNEQLLQQVDSKICHQEWDVLDEQFPNEEYLYQFCLLSAYYYALMVEGYGLLPNETINYLPSSQNLDWTIGVVLYPRLTVLQ

SEQ ID NO: 8 - protein, 360 aa, Legionella fallonii
ENASCDKHQCITVIDAGSTGSRVHIYAYDLDETNTPTNINEIWTKKIKPGFATIDPNYNTIDAYLTILLSGAPTQQGPVYFYATAGMRLLPASKQKLYYQELQRWFTQQTQWHFVEAKTITGNEEALYDWLSVNYHLGTLKSVQNKAIGVMDMGGASVQIVFPIQKNENIHGRSQVELNIYGQRINLFVHSFLGLGQTEMSHQFLNSTSCFTNDYPLPDGEMGLGNALSCEQEVSSLMNAVHGVSKTIQPVLAANSVDSWYAIGGLTNLADNKLFNFVNGQLTNQSLIEQADSQICHQQWSKLDAQYPNDDYIYGYCLFSSYFYALMVDGYGISPEQTVNYISPSINLDWTKGVVLHHLS

SEQ ID NO: 9 - protein, 388 aa, Gallaecimonas xiamenensis
ADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSVGDGRPEGRPGQQPGGVQHRQH

SEQ ID NO: 10 - protein, 358 aa, Legionella santicrucis
ESAECTEHQCMAVVDAGSTGSRAHIFAYDLDETNTPSQIHEIWSKKIKPGLATIEANQDTIDAYLTILFSGAPKQNIPVYFYATGGMRLIPQTKQKLYYKEIQNWFNQQSIWQLIEAKTITGNDEALYDWLAVNYHIGTLKSSTTEPIGVMDIGGASVQIAFPIPKDVETNKSSQVELDLYGKHLNLFVHSFLGLGQNEVTHQLLDTNSCFSEGYPLPGGNLGQGDAVICQQEVSSLINRVHKVQSEVQPTLATNRITMWYAIGGLTNLVESEPFQFQNNQITNQDLLQQANSLICHQQWEGLSTQFPDNEYIESFCLLPAFYYALMIDGYGISVTQPVKYISPRENLDWTTGVVLHH

SEQ ID NO: 11 - protein, 358 aa, Legionella quateirensis
ADNPCTNHQCIAVIDAGSTGSRLHVFSYDVDETRSPININEIWNKKIKPGFATIEPNSNTIDAYLTTLLSGAPNQQIPVYFYATAGMRLLPVSKQKKYYDELQHWFSQQSQWQLVEAKTITGNDEALYDWLSVNYHLGTLQKSLNQSVGVMDMGGASVQIVFPIQKNSELNTRSQVELDLYGQHYTLYVQSFLGLGQTEVAHQFLNSAPCFSNDYPLPDGESGQGNASACEQEVSRLMTEVHKVNNVIQPLLAANPVDSWYSIGGISNLADSSLFHFENSKLTNQNLLQQADSQVCHQQWDSLNSQFPDDEYVYQYCLFSAYYYALMVDGYGIVPDQSINYIPPAQNLDWTMGVVLHH

SEQ ID NO: 12 - protein, 358 aa, Legionella antarctica
GTVSCNEHQCIAVIDAGSTGSRIHVYAYDMDETNTPVHINEVWTKKIKPGFATIEPNYESIEAYLVTLLSGAPNQDIPVYFYATAGMRLLPASKQKLYYQELQRWFSQQHQWRLIDAKTITGNEEALYDWLSVNYHLDTLKSIQNKSVGVMDMGGASVQIVFPIQKNEEINSNSQVELDLYGHHVNLYVHSFLGLGQTEMSHQFLNSVSCFANDYPLPDGESGRGNAPSCKQEVSSLMNGVHGVNKTIQPVLTANPVDTWFAIGGLTNLADNKLFHFENGELTNQNLMQQADSQICHQQWDKLNSEFPNDDYVYEYCLLSAYYYALMVDGYGIYPEQTVNYVSSALNLDWTKGVVLHH

SEQ ID NO: 13 - protein, 360 aa, Legionella sp.
QSSPCEKQQCIAIVDAGSTGSRLHIYAYDLDVTKTPIHIKELWNKKITPGFARIEANTNTIDSYLTILLADSPIKKIPIYFYATAGMRLLPQMKQKKYYDELSSWFKKHSEWQLVEAKTITGNEEGLYDWLSVNYHLGNFESPTNKSIGVMDMGGASVQIVFPIAETPTINDKSQIQVNLYGRHYNLYVHSFLGLGQTEMSHQFLNSASCFVNNYPLPDGAEGQGNALSCEQEVSSLMNQVHGVNQLIQPLLAANPVDSWYSIGGISNLADSQFFNFQHSQLTNQDLLEQADSQICHQQWEDINNQYPDDDYVYQYCLFSAYYYALMVDGYGLYPEQTINYLPPQQNIDWTTGVVLHQQR

SEQ ID NO: 14 - protein, 380 aa, Paraglaciecola sp.
QSKKPCYIVYDAGSSGTRLYIYEQLGANLISHEGPKVSALADPIRYTHGKDLSDLDTITNEVASALDLIKLDGPLEEGKPKWSGFDWSKQCEAQSVKVFATAGMRIAEQINPADSLVLWQTLASKINAKVGHNIKVEARTLTGFEEGLFAWLSVKDVYPSGHYGMVEMGGASSQITFPCPQCSLANDAVKTVTVDGKPIQIYSYSYLGLGQNEAPYSLPFGVAGQVPKSCSYGVGANNPDWTIASCAVEIPITAKGSNTDIIDPYNYTKTGTKGTTNTLPAAHKNIPKWLLMGAFNYAKETDIDDFCFNQHDGYYNAFTACFNPIYLDKYLKTLNIQAEQSSKVDVSWTQGAVICQIENCLATITPPPVCRWMKSGCLAY

SEQ ID NO: 15 - protein, 370 aa, Beggiatoa sp.
ETNGPAACHIVYDAGSSGTRLYIYEQQDKAWLEHEGPKVSALADPVREIRGKHWKDAESVTIEVVSALDSIKQDGPVKGDKPAWKAFDWTKHCQIKSAGVYATAGMRLAEQANKERSVTLWASLKQKLAAKLGPTVDIEARTLTGYEEGLYAWLAVREQQPDDSFAIAEMGGASAQITFPCAKCDENDPDTKTVQLSGKHLQIYSYSFLGLGQDEASHSLGFPTSCANGIGTQQSGWKAQDCANQIQLTGSKGIRDPYDFDGEQRGAHEQPPIAQADSNRWFLTGAFNYMNASNIETYCLKDPKLSEEESFCFRSIYPEKYLETLKIPVNSPKMNVSWTQGAVICKANSCLEAATKTPPLCRWLDKGCLP

SEQ ID NO: 16 - protein, 385 aa, Betaproteobacteria sp.
EDAARYLAVIDAGSSGSRLHLYERTAAERSVAVKQLFEAKAGEALSSFERSPGDAGPRGIQPLLEQLLDRLALLNIERSAVHVHVLATAGMRRLDADNPAAAQVIYQSVRHTLSSAGVTARRIETLSGHLEGVYAWVDVNDLSGALASTPAGATVAVIEVGGASAQLAYSGEALAGAPVTAVRLNGRSHQVVSLTWLGLGQDEARRAMLNAARSRSNADEEHCYPDNRKAAGGLSAFDADGSRHLVARGRFSFEQCSEMYDAVIRPFDLPPLATAIRQSRRPLVGLASVYFSLADWQVIGQPGTLASTLRGQCEGEDAYGDKVIGFIRRDPKNRFAQTACANGTFIHALLFGPAGLRVDPAQVSAVARINGQPPSWARGFALLGR

SEQ ID NO: 17 - protein, 219 aa, Klebsiella aerogenes
KENKGFLNADTSPDSLAILPPPPTENSVAFLQDKADYETGRVLKNKERTELAAKDANYKNFGQAFSEAFGMVISAERTPVLYLLLSEVLQDSHDYAMRSAKDHYMRVRPFVLYKNPTCTPEKDDKMAKTGSYPSGHASFGWAAALVLAEINPARETEILKRGMDFGESRVVCGAHWQSDVDSGRIMGAAVVAALHANPRFANTLSEAKKEFAALSAKSE

SEQ ID NO: 18 - protein, 214 aa, Serratia rubidaea
AQTPGFLTPETAPDSLQILPPPPAENTTAFLNDKAAYETGKTLKDAQRQALAASDADYKNISAAFSAAFGEEISPEKTPALYALLQGVLQDSHDYAMRAAKDHYMRVRPFVVYKDSTCTPEKDKSMAKTGSYPSGHASFGWATALVLTEINPARETEILKRGYDFGQSRVVCGAHWQSDVDNGRLMGAAVVAALHGNQGFNDALAKAKAEFAKR

SEQ ID NO: 19 - protein, 221 aa, Herbaspirillum autotrophicum
KGAAGFLNGQTAPDSLAILPPPPAEDSVAFQADKAQYETGHVLRDPERVEQARQDADYRNFGHAFSAAFGMEISATHTPVLYQLLTEVLQDSHDYAMKAAKNHYKRVRPFVVYKDPTCTPGQDEKMALSGSYPSGHASFGWAAALVLAEINPARQTEILQRGYAFGKSRVICGAHWQSDVDNGQLMGAAVVAALHSNSQFAAALNAAKNEFAGLRAQDNKK

SEQ ID NO: 20 - protein, 219 aa, Acinetobacter apis
ADSNNSFLQLNQVPDSLKIVPAPPAFNSVDFLRDKAAYDQARSLVGTARWQQAAVDADLSDANIGKSFSEAFGVEISPTNTPVTYEILRKLRTDAGGYATKTAKEHYMRMRPFVFFNSHTCTPNDEASLSKNGAYPSGHTTIGWSSALILAEIRPDRQNEILERGYQYGQSRVICGVHWQSDVDAGRITGSAIVARLHADKDFAKTLEDAKREIQKKIK

SEQ ID NO: 21 - protein, 219 aa, Acinetobacter boissieri
LEQSNSFLQLAQVPDSLAIVPAPPAYNSVDFLRDKAAYDQAKSLVGTERWKVAAEDADLSDANIGKPFSQALGIEISPQNTPITYEILRKLRTDAGNYATKAAKEHYMRLRPFMFFNSHTCTPNDEANLSKNGAYPSGHTTIGWSSALVLAEIRPERQNEILQRGYEYGQSRVICGVHWQSDVDAGRITGSAIVARLHADKAFTKMLEEAKQEIQRKTK

SEQ ID NO: 22 - protein, 218 aa, Paraburkholderia bonniea
AGSDGFLTAETAPSSLAILPPPPAENTVAFLNDKANYEIGRSLKNDARVALAASDANCKNINAIFSSAFGREISEEKTPKLYRLLLGVLQDSHDHAMKSAKNHYMRVRPFVMYGDKTCTPEEDNKMKNTGSYPSGHASFGWAAALVLSEINPERTTEILRRGYDFGQSRVVCGAHWQSDVDNGRLMGAAVVADLHNNDKFIKALEAAKAEFKENTPGN

SEQ ID NO: 23 - protein, 220 aa, Pantoea sp.
LQSSGFLTAETAPDSLAILPPPPAENSVTFQSDKAQYEAGHVLRNVARTELASKDADYKNFGQAFSAAFGMEISESHTPVLYHLLSEVLQDSHDFAMRGAKDHYKRVRPFVIYKDSTCTPDKDKSMSTTGSYPSGHASFGWAAALILAEINPSRETEIIRRGYDFGESRVVCGAHWQSDVENGRIMGATVVASLHANAKFAELLSQAKNEFATLSAQDSK

SEQ ID NO: 24 - protein, 214 aa, Unknown
  environmental sample
GENSGFLSPETSPNSLEILPPPPSEGSIAFLNDKANYDIGHTLKNEGRLAQAVSDANYKNLTDAFSKAFGETISEQNTPALYSLLQNVLQDSHDFAMRAAKDHYKRVRPFVIYKDHTCTPAEDTKMAKTGSYPSGHASFGWATALVLSEINPARETEILKRGYEFGQSRVICGAHWQSDVDSGRIMGAAVVASLHNNSDFISALAKAKEEFRKK

SEQ ID NO: 25 - DNA, 1077 bp, Artificial Sequence
  codon-optimized nucleotide sequence
gatacgaatccgtgcgaaaaacattcttgcatcgcagttatcgatgcaggctctacaggctctcgcttacatatctatagctatgatacagataatacgaatacaccgatccatatcgaagaaatttggaataaaaaaattaaaccgggctttgcaagcatccaacctaatagcgttacgatcgatgcatatctgacgatgctgctggcagatgcaccgatccataatattccggtttatttttatgcaacggcaggcatgagacttcttcctcaaagccaacaaaaaaaatattatgatgaacttgaatattggtttcgccaacaatcacaatggcaacttgttgaagcaaaaacgatcacgggaaatgatgaagcactgtttgattggttagcagttaattataaactggatacgcttaaaagcgttcaaaataaatcagttggagttatggatatgggcggcgcaagcgttcaaattgtttttcctatgcctaaaaatgcagaaatctctaaacataatcaagttgaacttaatatctatggacaaaatattaatctgtatgttcatagctttctgggcctgggccaaacggaaatgtcacatcaatttcttaattctccgtcttgctttgcaaatgattatccgctgccggatggcgaaagcggccaaggcaatgcaccgagctgcaaagaagaagttacgtcacttatgaatagcgttcataaagttaatcaacaaatccaacctttactggcactgaatccggttaatgaatggtatagcatcggaggcatctctaatttagcaagctcacaactgtttcattttgaaaattcagaactgacgaatcaaagcctgcttcaacaaggagataatcaaatttgccatcaacaatgggatattctgaatggacaatatccggatgatgaatatctgtatcaatattgcctgctgtctagctattattatgcactgatggttgatggctatggcattaatcctaatcaaacgattcattatatcccgccggaacaaaatttagattggacgatcggcgttgttcttcatcgcgca

SEQ ID NO: 26 - DNA, 1098 bp, Artificial Sequence
  codon-optimized nucleotide sequence
atcgaacaagatccgtgccaagaaaaacaatgcatgattgttgttgatgcaggctctacaggaagccgccttcatttttatacgtatgatacagatgaaacgaatagcccgatcaatatcgaagaaatctggtctaaacgcattaaaccgggcatctcacatgttgaagcaaatcgcagctctatcgatgcatatcttgttacgctgctggcaggaacacctcatagcaatcatgttccggtttatttttatgcaacggcaggcatgcgcttactgccggcagcaaaacaaaaactgatctatcaagaaattaaattttggtttgcacaacaaaatcaatggagacttatggatgcaaaaacgatctatggctcacaagaagcactgtatgattggttagcagttaatcatcaactgggcacgttacaaagcgttcaaacgaaaagcgttggagttatggatatgggaggcgcatcagttcaaatcgtttttccggttcaaaataatccggatcttaaaaataatacattagcagaatttaatctgtatggccaacatattcgcctggcagttcgcagctttcttggcttaggccaaacggaaatgtcacatcaatttctgaattatgcaccgtgctttgcaaataattatccgctgccggatggctttctgggcgaaggagatgcactgtcttgcgttgaagaagttgcaagcctgatgaatggagttcatagcgttaataaagaaatccaacctctgctggcagcaaatcctgttaatatttggtatgcaatcggcggactgagctttcttgcagataattcagttatccattttaataattatcaacttacgaatgaacaacttcttcaacaagttgattctaaaatctgccatcaagaatgggatgttttagatgaacaatttcctaatgaagaatatctgtatcaattttgcctgctgagcgcatattattatgcactgatggttgaaggctatggacttcttcctaatgaaacgattaattatttaccgtctagccaaaatttagattggacgatcggcgttgttctgtatcctcgcctgacggttttacaa

SEQ ID NO: 27 - DNA, 1080 bp, Artificial Sequence
  codon-optimized nucleotide sequence
gaaaatgcaagctgcgataaacatcaatgcattacagttatcgatgcaggaagcacgggctctcgcgttcatatctatgcatatgatttagatgaaacgaatacaccgacgaatattaatgaaatttggacgaaaaaaattaaaccgggctttgcaacgatcgatccgaattataatacgatcgatgcatatcttacgatcctgctgtcaggcgcacctacacaacaaggaccggtttatttttatgcaacagcaggcatgagactgcttccggcatctaaacaaaaactgtattatcaagaacttcaacgctggtttacacaacaaacgcaatggcattttgttgaagcaaaaacgatcacgggaaatgaagaagcactgtatgattggctgagcgttaattatcatttaggcacgcttaaatcagttcaaaataaagcaatcggagttatggatatgggaggcgcaagcgttcaaatcgtttttcctattcaaaaaaatgaaaatatccatggccgctctcaagttgaacttaatatctatggccaacgcattaatctgtttgttcatagctttctgggcttaggccaaacagaaatgtcacatcaatttctaaattctacgtcttgctttacgaatgattatcctcttccggatggagaaatgggactgggcaatgcactgtcttgcgaacaagaagttagctcacttatgaatgcagttcatggagtttctaaaacgatccaaccggttcttgcagcaaatagcgttgatagctggtatgcaatcggcggccttacgaatctggcagataataaactgtttaattttgttaatggccaactgacgaatcaaagcctgattgaacaagcagatagccaaatctgccatcaacaatggtctaaacttgatgcacaatatcctaatgatgattatatctatggctattgcctgttttctagctatttttatgcactgatggttgatggctatggaatctcaccggaacaaacggttaattatatcagcccgagcattaatttagattggacgaaaggagttgttcttcatcatctgagc

SEQ ID NO: 28 - DNA, 1164 bp, Artificial Sequence
  codon-optimized nucleotide sequence
gcagataaaccggcatgccgcgcagtttttgatgcaggctcatcaggaacgagactgtttctgtatgttcaacaagaaggcggctggcaaccgtggccgggcgcaaaactgggcgcacttgcagatccggttcgccaacatcaaggacctaaagcaatggcagcattagcagcagatcttgcaaattctcttgaagcacttcgccaagatggacctacgaatagcgaaggccaacctcgctggcaaggctttgattggcaacaagcatgccgcttacaatctgttagcgttcttgcaacggcaggaatgagacttgcagaacaacaagatccggttgcatctgttgcactgtggcaagaagttcaacaagcactggcaggattactgggccctgcagttgcaatcgatgcacgcacactgacgggctttgaagaaggattttatgcatggttagcagcacgcgcacgccttggacgctctgatatgggaatcgttgaaatgggcggagcatcatctcaagttgcatttccgtgccctgcatgcgatcgcgcagatgatgcagttcaacaagttagacttgaaggccaaacgctggatttttatagctatagctttctgggattaggacaagatgaagcaccgaaagttttaggcgttcctcatgcatgccgctatggagcaggagttaaagatcctaattggcaattaggacaatgccaagcacaacttcctcttacgttaggccgcgcacttaaagatccgtataattttcaaggccaaggccaaggcgggtatcgccaagttccgacggaaaaagcaggagttaaagattggttattaacgggcgcatttcaatataatacacctgatcaattagatagctgctgcttaaatcaaggccaatgctatcaaccgtctacggcatgctttcgcgttgcatatttacctctgtatcttgatagccttggagttgatcctcattctcctgatttagatgtttcatggacactgggagcagttctgtgccgcaatgaacaatgcctggcaccgtctaaacctccgtgccgctggagaaaagcaggctgcctggcaccgagcgttggcgatggacgcccggaaggaagaccgggacaacaaccgggcggcgttcaacatagacaacat

SEQ ID NO: 29 - DNA, 1074 bp, Artificial Sequence
  codon-optimized nucleotide sequence
gaaagcgcagaatgcacggaacatcaatgcatggcagttgttgatgcaggctctacaggctctcgcgcacatatctttgcatatgatctggatgaaacgaatacgccgagccaaatccatgaaatttggtctaaaaaaattaaaccgggcctggcaacgattgaagcaaatcaagatacgatcgatgcatatctgacgatcctgtttagcggagcacctaaacaaaatattccggtttatttttatgcaacgggaggaatgagactgattcctcaaacgaaacaaaaactgtattataaagaaatccaaaattggtttaatcaacaatctatctggcaactgattgaagcaaaaacgatcacgggcaatgatgaagcactgtatgattggttagcagttaattatcatatcggcacgcttaaaagctctacgacagaacctatcggcgttatggatattggaggcgcatcagttcaaatcgcatttcctatccctaaagatgttgaaacgaataaaagcagccaagttgaacttgatctgtatggaaaacatcttaatctgtttgttcattcttttctgggactgggccaaaatgaagttacacatcaacttctggatacgaatagctgcttttcagaaggctatccgctgccgggaggcaatcttggccaaggagatgcagttatttgccaacaagaagttagctcactgattaatagagttcataaagttcaatcagaagttcaaccgacgcttgcaacgaatcgcattacaatgtggtatgcaatcggcggcctgacgaatttagttgaaagcgaaccgtttcaatttcaaaataatcaaattacgaatcaagatctgctgcaacaagcaaatagccttatttgccatcaacaatgggaaggactgtctacacaatttccggataatgaatatatcgaatccttttgcttacttccggcattttattatgcacttatgatcgatggctatggcattagcgttacacaaccggttaaatatatctcacctagagaaaatttagattggacgacaggcgttgttcttcatcat

SEQ ID NO: 30 - DNA, 1074 bp, Artificial Sequence
  codon-optimized nucleotide sequence
gcagataatccgtgcacgaatcatcaatgcatcgcagttatcgatgcaggctctacaggctctcgcttacatgtttttagctatgatgttgatgaaacgcgctcacctattaatattaatgaaatctggaataaaaaaattaaaccgggctttgcaacgatcgaaccgaatagcaatacgatcgatgcatatctgacgacactgctgtcaggagcaccgaatcaacaaatcccggtttatttttatgcaacggcaggcatgcgcctgctgccggtttctaaacaaaaaaaatattatgatgaacttcaacattggtttagccaacaaagccaatggcaacttgttgaagcaaaaacgattacgggaaatgatgaagcactgtatgattggctgagcgttaattatcatttaggaacgttacaaaaatctctgaatcaaagcgttggagttatggatatgggcggcgcaagcgttcaaatcgtttttcctatccaaaaaaattcagaacttaatacacgctcacaagttgaactggatctgtatggacaacattatacgctgtatgttcaatcttttctgggccttggccaaacggaagttgcacatcaatttcttaattcagcaccgtgcttttctaatgattatcctcttccggatggagaaagcggccaaggaaatgcaagcgcatgcgaacaagaagtttctagactgatgacagaagttcataaagttaataatgttatccaacctctgctggcagcaaatccggttgatagctggtatagcatcggaggcatctctaatcttgcagatagctcactgtttcattttgaaaattctaaactgacgaatcaaaatttacttcaacaagcagatagccaagtttgccatcaacaatgggatagcttaaattcacaatttccggatgatgaatatgtttatcaatattgcctgtttagcgcatattattatgcacttatggttgatggctatggcattgttccggatcaatctattaattatatccctcctgcacaaaatttagattggacgatgggcgttgttcttcatcat

SEQ ID NO: 31 - DNA, 1074 bp, Artificial Sequence
  codon-optimized nucleotide sequence
ggcacagttagctgcaatgaacatcaatgcatcgcagttatcgatgcaggctctacgggctctcgcatccatgtttatgcatatgatatggatgaaacgaatacaccggttcatattaatgaagtttggacgaaaaaaattaaaccgggctttgcaacgatcgaacctaattatgaaagcattgaagcatatcttgttacactgctgtcaggcgcaccgaatcaagatattccggtttatttttatgcaacagcaggcatgcgcttacttccggcatctaaacaaaaactgtattatcaagaacttcaacgctggtttagccaacaacatcaatggagactgatcgatgcaaaaacgatcacgggaaatgaagaagcactgtatgattggctgtcagttaattatcatttagatacgcttaaatctatccaaaataaaagcgttggagttatggatatgggaggagcaagcgttcaaatcgtttttcctatccaaaaaaatgaagaaatcaattctaatagccaagttgaactggatctgtatggacatcatgttaatctgtatgttcatagctttctgggcctgggacaaacggaaatgtcacatcaatttcttaatagcgttagctgctttgcaaatgattatcctctgccggatggcgaatccgggcggggcaatgcaccgtcttgcaaacaagaagttagctcactgatgaatggcgttcatggcgttaataaaacgatccaacctgttcttacagcaaatccggttgatacgtggtttgcaatcggaggcctgacgaatctggcagataataaactttttcattttgaaaatggcgaacttacgaatcaaaatctgatgcaacaagcagattcacaaatttgccatcaacaatgggataaacttaattcagaatttcctaatgatgattatgtttatgaatattgcctgctgagcgcatattattatgcactgatggttgatggctatggaatctatccggaacaaacggttaattatgtttctagcgcacttaatttagattggacgaaaggagttgttcttcatcat

SEQ ID NO: 32 - DNA, 1080 bp, Artificial Sequence
  codon-optimized nucleotide sequence
caaagctcaccgtgcgaaaaacaacaatgcatcgcaattgttgatgcaggctctacgggctctcgcttacatatctatgcatatgatttagatgttacgaaaacgccgatccatattaaagaactgtggaataaaaaaattacaccgggctttgcacgcatcgaagcaaatacgaatacgatcgatagctatctgacgatcctgcttgcagattcacctattaaaaaaatccctatctatttttatgcaacggcaggcatgagacttcttcctcaaatgaaacaaaaaaaatattatgatgaactgagctcttggtttaaaaaacattcagaatggcaactggttgaagcaaaaacgatcacaggcaatgaagaaggactgtatgattggctgagcgttaattatcatttaggcaattttgaaagcccgacgaataaatctatcggcgttatggatatgggaggagcatcagttcaaattgtttttccgatcgcagaaacgccgacgattaatgataaaagccaaatccaagttaatctgtatggccgccattataatctgtatgttcatagctttctgggactgggccaaacggaaatgtcacatcaatttctgaatagcgcaagctgctttgttaataattatcctctgccggatggagcagaaggacaaggaaatgcactgtcatgcgaacaagaagtttctagcctgatgaatcaagttcatggagttaatcaactgatccaaccgctgctggcagcaaatccggttgatagctggtatagcatcggcggcatctctaatcttgcagatagccaattttttaattttcaacattctcaacttacgaatcaagatttacttgaacaagcagattcacaaatttgccatcaacaatgggaagatattaataatcaatatccggatgatgattatgtttatcaatattgcctgtttagcgcatattattatgcacttatggttgatggctatggcctgtatccggaacaaacgatcaattatcttcctccgcaacaaaatatcgattggacgacaggcgttgttcttcatcaacaacgc

SEQ ID NO: 33 - DNA, 1140 bp, Artificial Sequence
  codon-optimized nucleotide sequence
caatctaaaaaaccgtgctatattgtttatgatgcaggctctagcggaacgagactgtatatctatgaacaactgggcgcaaatcttatttctcatgaaggaccgaaagtttcagcactggcagatcctattcgctatacacatggcaaagatctgtcagatttagatacgatcacgaatgaagttgcatcagcacttgatctgattaaactggatggcccgttagaagaaggcaaacctaaatggtcaggctttgattggtctaaacaatgcgaagcacaatctgttaaagtttttgcaacagcaggaatgagaatcgcagaacaaatcaatccggcagatagccttgttctgtggcaaacgttagcatctaaaattaatgcaaaagttggacataatattaaagttgaagcacgcacgctgacgggctttgaagaaggactgtttgcatggctgagcgttaaagatgtttatccgagcggccattatggaatggttgaaatgggaggcgcatcaagccaaattacgtttccgtgccctcaatgctctcttgcaaatgatgcagttaaaacggttacggttgatggaaaaccgattcaaatttatagctatagctatctgggcctgggacaaaatgaagcaccgtatagcttaccttttggagttgcaggccaagttccgaaaagctgctcatatggcgttggagcaaataatccggattggacgatcgcatcatgcgcagttgaaatcccgatcacggcaaaaggctctaatacggacataatcgatccgtataattatacgaaaacaggaacgaaaggaacgacgaatacacttcctgcagcacataaaaatattcctaaatggttacttatgggcgcatttaattatgcaaaagaaacggacatagatgatttttgctttaatcaacatgatggctattataatgcatttacagcatgctttaatcctatctatttagataaatatcttaaaacgctgaatattcaagcagaacaatcttctaaagttgatgtttcttggacacaaggcgcagttatctgccaaatcgaaaattgcctggcaacgatcacacctccgccggtttgccgctggatgaaaagcggctgcctggcatat

SEQ ID NO: 34 - DNA, 1110 bp, Artificial Sequence
  codon-optimized nucleotide sequence
gaaacgaatggccctgcagcatgccatattgtttatgatgcaggctctagcggcacgagactgtatatctatgaacaacaagataaagcatggttagaacatgaaggacctaaagtttcagcacttgcagatccggttcgcgaaattagaggcaaacattggaaagatgcagaatcagttacgatcgaagttgttagcgcacttgattctattaaacaagatggaccggttaaaggagataaaccggcatggaaagcatttgattggacgaaacattgccaaattaaatcagcaggagtttatgcaacggcaggaatgagactggcagaacaagcaaataaagaacgctcagttacactgtgggcatcacttaaacaaaaacttgcagcaaaacttggcccgacagttgatatagaagcacgcacgctgacgggctatgaagaaggcctgtatgcatggctggcagttcgcgaacaacaaccggatgattcatttgcaatcgcagaaatgggaggcgcatctgcacaaattacgtttccgtgcgcaaaatgcgatgaaaatgatccggatacgaaaacggttcaactgagcggaaaacatcttcaaatttatagctatagctttctgggactgggccaagatgaagcatctcatagccttggctttcctacgtcatgcgcaaatggaatcggcacacaacaatcaggatggaaagcacaagattgcgcaaatcaaattcaacttacaggctctaaagggatacgcgatccgtatgattttgatggcgaacaacgcggcgcacatgaacaacctccgatcgcacaagcagattctaatcgctggtttctgacgggcgcatttaattatatgaatgcatctaatatcgaaacgtattgccttaaagatcctaaactgtctgaagaagaatctttttgctttcgctcaatttatcctgaaaaatatttagaaacgcttaaaatccctgttaatagccctaaaatgaatgtttcttggacacaaggagcagttatctgcaaagcaaatagctgcttagaagcagcaacgaaaacgccgcctctgtgccgctggttagataaaggctgcttacct

SEQ ID NO: 35 - DNA, 1155 bp, Artificial Sequence
  codon-optimized nucleotide sequence
gaagatgcagcacgctatctggcagttatcgatgcaggctcatcaggctctcgcttacatctgtatgaaagaacggcagcagaacgctctgttgcagttaaacaactttttgaagcaaaagcaggagaagcactgtctagctttgaacgctcaccgggcgatgcaggaccgagaggtattcaacctctgctggaacaacttcttgatagacttgcattacttaatatcgaaagatcagcagttcatgttcatgttctggcaacggcaggaatgagacgccttgatgcagataatcctgcagcagcacaagttatctatcaaagcgttcgccatacactgtctagcgcaggagttacggcacgccgcatcgaaacactgagcggacatttagaaggcgtttatgcatgggttgatgttaatgatctgagcggcgcactggcatctacaccggcaggagcaacggttgcagttatcgaagttggaggcgcaagcgcacaattagcatatagcggcgaagcattagcaggcgcaccggttacggcagttcgccttaatggccgctcacatcaagttgtttctctgacgtggttaggactgggccaagatgaagcacgccgcgcaatgctgaatgcagcacgctctcgctcaaatgcagatgaagaacattgctatccggataatagaaaagcagcaggcggactgagcgcatttgatgcagatggatctagacatcttgttgcacgcggacgcttttcttttgaacaatgctcagaaatgtatgatgcagttattcgcccgtttgatttacctccgctggcaacggcaatcagacaatctagacgccctcttgttggattagcaagcgtttattttagcctggcagattggcaagttatcggacaaccgggaacactggcatctacactgagaggccaatgcgaaggagaagatgcatatggcgataaagttattggatttattcgccgcgatcctaaaaatagatttgcacaaacagcatgcgcaaatggaacgtttattcatgcacttctgtttggacctgcaggccttcgcgttgatcctgcacaagtttcagcagttgcaagaattaatggccaacctccgtcatgggcaagaggctttgcattactgggccgc

SEQ ID NO: 36 - DNA, 657 bp, Artificial Sequence
  codon-optimized nucleotide sequence
aaagaaaataaaggctttcttaatgcagatacgagcccggatagcctggcaatcttacctcctccgcctacggaaaatagcgttgcatttcttcaagataaagcagattatgaaacaggacgcgttcttaaaaataaagaacgcacggaactggcagcaaaagatgcaaattataaaaattttggacaagcatttagcgaagcatttggaatggttatctcagcagaacgcacaccggttctgtatttactgctgtctgaagttttacaagattcacatgattatgcaatgcgctctgcaaaagatcattatatgagagttagaccgtttgttctgtataaaaatccgacgtgcacacctgaaaaagatgataaaatggcaaaaacaggctcatatccgtcaggacatgcatcttttggctgggcagcagcactggttctggcagaaattaatccggcacgcgaaacggaaatccttaaacgcggaatggattttggagaatctagagttgtttgcggcgcacattggcaatctgatgttgattcaggccgcatcatgggcgcagcagttgttgcagcacttcatgcaaatcctcgctttgcaaatacactgagcgaagcaaaaaaagaatttgcagcacttagcgcaaaatcagaa

SEQ ID NO: 37 - DNA, 642 bp, Artificial Sequence
  codon-optimized nucleotide sequence
gcacaaacaccgggctttcttacacctgaaacggcaccggatagccttcaaattcttcctccgcctcctgcagaaaatacgacggcatttctgaatgataaagcagcatatgaaacaggcaaaacacttaaagatgcacaacgccaagcactggcagcaagcgatgcagattataaaaatattagcgcagcatttagcgcagcatttggagaagaaattagccctgaaaaaacacctgcactgtatgcattacttcaaggagttttacaagattcacatgattatgcaatgagagcagcaaaagatcattatatgagagttagaccgtttgttgtttataaagattctacgtgcacgccggaaaaagataaatctatggcaaaaacaggctcatatccgtcaggacatgcatcttttggatgggcaacggcactggttctgacagaaatcaatccggcacgcgaaacggaaatccttaaacgcggctatgattttggacaatctcgcgttgtttgcggcgcacattggcaatctgatgttgataatggccgcttaatgggcgcagcagttgttgcagcacttcatggaaatcaaggctttaatgatgcactggcaaaagcaaaagcagaatttgcaaaacgc

SEQ ID NO: 38 - DNA, 663 bp, Artificial Sequence
  codon-optimized nucleotide sequence
aaaggcgcagcaggctttcttaatggacaaacggcacctgattcactggcaatcttacctcctccgcctgcagaagatagcgttgcatttcaagcagataaagcacaatatgaaacaggccatgttctgagagatccggaacgcgttgaacaagcacgccaagatgcagattatcgcaattttggacatgcatttagcgcagcatttggaatggaaatctctgcaacacatacaccggttctgtatcaactgttaacagaagttttacaagattctcatgattatgcaatgaaagcagcaaaaaatcattataaacgcgttagaccgtttgttgtttataaagatcctacgtgcacaccgggccaagatgaaaaaatggcactgtcaggatcatatccgagcggacatgcatcttttggctgggcagcagcattagttctggcagaaattaatccggcacgccaaacggaaatccttcaacgcggctatgcatttggcaaatctagagttatctgcggcgcacattggcaaagcgatgttgataatggacaactgatgggcgcagcagttgttgcagcacttcattctaatagccaatttgcagcagcacttaatgcagcaaaaaatgaatttgcaggacttcgcgcacaagataataaaaaa

SEQ ID NO: 39 - DNA, 657 bp, Artificial Sequence
  codon-optimized nucleotide sequence
gcagattctaataatagctttcttcaactgaatcaagttcctgatagccttaaaattgttccggcacctccggcatttaattcagttgattttcttcgcgataaagcagcatatgatcaagcacgctcactggttggaacagcacgctggcaacaagcagcagttgatgcagatctgagcgatgcaaatatcggcaaatcatttagcgaagcatttggcgttgaaatctcacctacgaatacaccggttacgtatgaaatccttcgcaaacttagaacggatgcaggaggctatgcaacgaaaacggcaaaagaacattatatgagaatgcgcccgtttgttttttttaatagccatacgtgcacacctaatgatgaagcatctctgtctaaaaatggcgcatatccgtcaggacatacgacgatcggctggtcatcagcacttatcctggcagaaattagaccggatcgccaaaatgaaattcttgaaagaggctatcaatatggacaatctcgcgttatttgcggagttcattggcaatctgatgttgatgcaggccgcatcacgggaagcgcaattgttgcacgcttacatgcagataaagattttgcaaaaacactggaagatgcaaaacgcgaaatccaaaaaaaaattaaa

SEQ ID NO: 40 - DNA, 657 bp, Artificial Sequence
  codon-optimized nucleotide sequence
cttgaacaatctaatagctttcttcaactggcacaagttcctgatagcctggcaattgttccggcacctccggcatataattcagttgattttctgagagataaagcagcatatgatcaagcaaaatcactggttggaacagaacgctggaaagttgcagcagaagatgcagatctgagcgatgcaaatatcggaaaaccgtttagccaagcattaggcatcgaaatctcacctcaaaatacaccgattacgtatgaaatcctgcgcaaacttcgcacggatgcaggcaattatgcaacgaaagcagcaaaagaacattatatgagacttagaccgtttatgttttttaatagccatacgtgcacacctaatgatgaagcaaatctgtctaaaaatggcgcatatccgtcaggacatacgacgatcggctggtctagcgcactggttcttgcagaaattcgcccggaacgccaaaatgaaattcttcaacgcggctatgaatatggacaatctcgcgttatttgcggagttcattggcaatcagatgttgatgcaggccgcatcacgggctctgcaattgttgcacgcttacatgcagataaagcatttacgaaaatgttagaagaagcaaaacaagaaatccaacgcaaaacgaaa

SEQ ID NO: 41 - DNA, 654 bp, Artificial Sequence
  codon-optimized nucleotide sequence
gcaggctctgatggatttcttacggcagaaacggcaccgagctcactggcaattcttcctccgcctccggcagaaaatacggttgcatttctgaatgataaagcaaattatgaaatcggacgctcacttaaaaatgatgcacgcgttgcattagcagcatcagatgcaaattgcaaaaatatcaatgcaatctttagctcagcatttggccgcgaaatctctgaagaaaaaacacctaaactgtatagactgttactgggagttttacaagattcacatgatcatgcaatgaaaagcgcaaaaaatcattatatgcgcgttcgcccgtttgttatgtatggagataaaacgtgcacaccggaagaagataataaaatgaaaaatacaggatcatatccgtcaggacatgcatcttttggctgggcagcagcactggttctgagcgaaattaatccggaacgcacgacggaaatccttagacgcggctatgattttggacaatctagagttgtttgcggcgcacattggcaaagcgatgttgataatggccgccttatgggcgcagcagttgttgcagatcttcataataatgataaatttatcaaagcacttgaagcagcaaaagcagaatttaaagaaaatacaccgggcaat

SEQ ID NO: 42 - DNA, 660 bp, Artificial Sequence
  codon-optimized nucleotide sequence
cttcaaagctcaggctttcttacggcagaaacagcaccggatagcctggcaatccttcctccgcctccggcagaaaatagcgttacgtttcaatctgataaagcacaatatgaagcaggacatgttcttcgcaatgttgcacgcacggaactggcatctaaagatgcagattataaaaattttggacaagcatttagcgcagcatttggaatggaaattagcgaatcacatacaccggttctgtatcatctgctgtctgaagttttacaagatagccatgattttgcaatgagaggcgcaaaagatcattataaacgcgttagaccgtttgttatctataaagattcaacgtgcacacctgataaagataaatctatgtctacgacgggctcatatccgtcaggccatgcatcttttggctgggcagcagcacttatcctggcagaaattaatccgagccgcgaaacggaaattattcgccgcggctatgattttggagaatctagagttgtttgcggcgcacattggcaaagcgatgttgaaaatggacgcatcatgggagcaacagttgttgcatcacttcatgcaaatgcaaaatttgcagaactgctgtctcaagcaaaaaatgaatttgcaacactgagcgcacaagattctaaa

SEQ ID NO: 43 - DNA, 642 bp, Artificial Sequence
  codon-optimized nucleotide sequence
ggagaaaattcaggctttctgtcaccggaaacgtcacctaatagcctggaaatccttccgcctcctccgagcgaaggctctatcgcatttctgaatgataaagcaaattatgacatcggacatacacttaaaaatgaaggacgccttgcacaagcagttagcgatgcaaattataaaaatcttacggatgcattttctaaagcatttggagaaacgatctctgaacaaaatacacctgcactgtatagcttacttcaaaatgttcttcaagatagccatgattttgcaatgcgcgcagcaaaagatcattataaacgcgttagaccgtttgttatctataaagatcatacgtgcacaccggcagaagatacgaaaatggcaaaaacgggctcatatccgtcaggccatgcttcttttggctgggcaacagcactggttctgtctgaaattaatccggcacgcgaaacagaaatccttaaacgcggatatgaatttggacaatctagagttatctgcggcgcacattggcaatcagatgttgattcaggccgcatcatgggagcagcagttgttgcatctttacataataatagcgattttattagcgcactggcaaaagcaaaagaagaatttcgcaaaaaa

SEQ ID NO: 44 - protein, 388 aa, Gallaecimonas mangrovi
MFKRAVVVCCAFFALLSASAHAAKPACQMVFDAGSSGTRLYLYQATGHGWREFAGPTSPALADPVRGINGQTADDIPKVAKALGDSIKAFRHNGPKGKEGKPAWHGYDWQHRCALKSVSVLATAGMRLAEQQQLGLSQVLWMAVRQQVQFAVGPKVRVKARTISGFEEGLYGWLSLTKAKKTLNIGMVEMGGASSQVAFPCLYCRHDDDAIRQIQIDGKTLPFYSYSFLGLGQDEAAKVYKLPKRCQYGIGAVIPGWSLEQCSERLLLTHDGELKDPYNLNDKGRVGRRRDVPVALAQVSQWYLTGAFNYLESDQVQRCCLTKGACYHPQTACFRAAYLPKYLHDLQIPASAKPAKSNWTEGAMICQSQHCLAKAKPQQCRWSKFGCL

SEQ ID NO: 45 - protein, 382 aa, Gallaecimonas pentaromativorans
MRPLLFLLLSLTASPLLAKPACQLMFDAGSSGTRLFLFQQKGGQLVSQRGPKIAALADPVRGIRGKTAEDIPATAMALAKALEDFRHDGPAGEGGKPRWRGFDWRERCRLQAVSVLATAGMRLAEQEAPGLSQTLWLAVRQQLQLVVGPKVQVEARTLSGFEEGLYAWLARYQKRQRRDFGIVEMGGASSQVAFPCPACSLDDDALHQVVIGGKAVPFYSYSFLGLGQDLAPGYLGLPDPCRFGAGKGNPRWRLSQCQAQLLLTQGPAIRDPANYQGGIKGTERQVPVAKAGVDKWYLTGAFKYLEADQVARCCQGSGQCYQPETACFRAAYLPLLLEKLGVPATAKVKDSSWTLGAALCRQENCLAKAGVLACRWSKNGCL

SEQ ID NO: 46 - protein, 368 aa, Legionella pneumophila
MDTNPCEKHSCIAVIDAGSTGSRLHIYSYDTDDTNTPIHIEEIWNKKIKPGFASIQPNSVTIDAYLTMLLADAPIHNIPVYFYATAGMRLLPQSQQKKYYDELDYWFRQQSQWQLVEAKTITGNDEALFDWLAVNYKLDTLKSVQNKSVGVMDMGGASVQIVFPMPKNAEISKHNQVELNIYGQNINLYVHSFLGLGQTEMSHQFLNSPSCFANDYPLPDGESGQGNAPSCKEEVTSLMNSVHKVNQQIQPLLALNPVNEWYSIGGISNLASSQLFHFENSELTNQSLLQQGDNQICHQQWDILNGQYPDDEYLYQYCLLSSYYYALMVDGYGINPNQTIHYIPPEQNLDWTIGVVLHRALEHHHHHH

SEQ ID NO: 47 - protein, 249 aa, Shimwellia blattae
MKKRVLAVCFAALFSSQALALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPAFQQQLQKAKAEFAQHQKK

SEQ ID NO: 48 - protein, 414 aa, Aspergillus fumigatus
MFTKKSLVTLLGGLSVALAQTSSEQYPSLSEIEAAQATVLPHSPVSNVKGLAFDRFVNIWLENTDFDKAAADAHMSALAKKGLLLTNFWAVTHPSEPNYCAAAGGDTFGMDNDNFNQVPANVSTIADLFDTKNIAWGEYQEHLPYPGYQGFRYPESGPNDYVRKHNPAILFDSVTQDATRLRQIKNFTSFYDDLKNHRLPQHMFITPNMTNDAHDTNITFAATWSWNFLSELLENDYFTKDTLILLTFDENGTYKLGNKIYSILLGGAVPKHLIGKEDNTYYNHYSVIASLSANWGLPSLGRWDCGANLLSFIAEKTGYVNWEVDTSNLYLNHSYPGPLSTSSHTPNWPVPLTKGSCSAGHGILKAVRKTYKDMAPTYNYTSPVPYDAASKTNVGVKYTRKLVSLLDQSLTHEY

SEQ ID NO: 49 - protein, 426 aa, Homo sapiens
MAGLGFWGHPAGPLLLLLLLVLPPRALPEGPLVFVALVFRHGDRAPLASYPMDPHKEVASTLWPRGLGQLTTEGVRQQLELGRFLRSRYEAFLSPEYRREEVYIRSTDFDRTLESAQANLAGLFPEAAPGSPEARWRPIPVHTVPVAEDKLLRFPMRSCPRYHELLREATEAAEYQEALEGWTGFLSRLENFTGLSLVGEPLRRAWKVLDTLMCQQAHGLPLPAWASPDVLRTLAQISALDIGAHVGPPRAAEKAQLTGGILLNAILANFSRVQRLGLPLKMVMYSAHDSTLLALQGALGLYDGHTPPYAACLGFEFRKHLGNPAKDGGNVTVSLFYRNDSAHLPLPLSLPGCPAPCPLGRFYQLTAPARPPAHGVSCHGPYEAAIPPAPVVPLLAGAVAVLVALSLGLGLLAWRPGCLRALGGPV

SEQ ID NO: 50 - protein, 472 aa, Arabidopsis thaliana
MTAKRGIGRHESLADKIQRHRGIILVISVPIVLIGLVLLLMPGRSISDSVVEEYSVHNRKGGPNSRGPKNYAVIFDAGSSGSRVHVYCFDQNLDLIPLGNELELFLQLKPGLSAYPTDPRQAANSLVSLLDKAEASVPRELRPKTHVRVGVTAGLRTLGHDASENILQAVRELLRDRSMLKTEANAVTVLDGTQEGSYQWVTINYLLRNLGKPYSDTVGVVDLGGGSVQMAYAISEEDAASAPKPLEGEDSYVREMYLKGRKYFLYVHSYLHYGLLAARAEILKVSEDSENPCIVAGYDGTYKYGGKEFKAPASQSGASLDECRRITINALKVNDTLCTHMKCTFGGVWNGGRGGGQKNMFVASFFFDRAAEAGFVDPKQPVATVRPMDFEKAAKKACSMKLEEGKSTFPLVEEENLPYLCMDLVYQYTLLIDGFGLEPSQTITLVKKVKYGDQAVEAAWPLGSAIEAVSSP